(19) United States
(12) Patent Application Publication
(10) Pub. No.: US 2026/0041695 A1
(43) Pub. Date: Feb. 12, 2026

(54) COMBINATION THERAPIES FOR TREATMENT OF BREAST CANCER

(71) Applicant: Genentech, Inc., South San Francisco, CA (US)

(72) Inventors: Jennifer O'Hara Lauchle, Larkspur, CA (US); Michael Joseph Mamounas, San Francisco, CA (US); Jennifer Lee Schutzman, Belmont, CA (US); Lori Sickels Friedman, San Carlos, CA (US); Jian Huang, Millbrae, CA (US)

(73) Assignee: Genentech, Inc., South San Francisco, CA (US)

(21) Appl. No.: 19/362,148

(22) Filed: Oct. 17, 2025

Related U.S. Application Data
(63) Continuation of application No. 17/831,617, filed on Jun. 3, 2022.
Application No. 17/831,617 is a continuation of application No. PCT/US2020/062673, filed on Dec. 1, 2020.
(60) Provisional application No. 62/943,185, filed on Dec. 3, 2019.
(60) Provisional application No. 62/946,400, filed on Dec. 10, 2019.
(60) Provisional application No. 63/014,965, filed on Apr. 24, 2020.

(51) Int. Cl. A61K 31/553; A61K 31/4196; A61K 31/519; A61K 31/565; A61P 35/00

(52) U.S. Cl. CPC A61K 31/553; A61K 31/4196; A61K 31/519; A61K 31/565; A61P 35/00

(57) ABSTRACT

Provided are combination therapies comprising a PI3K inhibitor (e.g., inavolisib), a CDK4/6 inhibitor (e.g., palbociclib), and fulvestrant; and methods of treating hormone receptor positive and HER2 negative (HR+/HER2−) locally advanced or metastatic breast cancer in a patient (preferably a patient with a PIC3CA mutant patient) comprising administering a therapeutically effective amount of inavolisib, or a pharmaceutically acceptable salt thereof, a CDK4/6 inhibitor (e.g., palbociclib), and fulvestrant or letrozole.

### CROSS-REFERENCE TO RELATED APPLICATIONS

This application claims the benefit of U.S. Provisional Patent Application No. 62/943,185, filed Dec. 3, 2019; U.S. Provisional Patent Application No. 62/946,400, filed Dec. 10, 2019; and U.S. Provisional Patent Application No. 63/014,965, filed Apr. 24, 2020; the contents of which are incorporated by reference herein in their entireties.

### FIELD OF THE INVENTION

The invention relates generally to treatment of PIK3CA-mutant cancer patients by administering a PI3K inhibitor, inavolisib (a.k.a. GDC-0077), in combination with a CDK4/6 inhibitor and an endocrine therapy for the treatment of breast cancers.

### BACKGROUND

Globally, breast cancer is the second most common invasive malignancy and the most common cause of cancer-related mortality in women, with a 5-year survival rate following metastatic diagnosis of approximately 15%.

Phosphatidylinositol 3-kinase (PI3K) is a lipid kinase that upon activation by growth factor receptors and integrins regulates cell proliferation, survival, and migration. PI3K catalyzes the phosphorylation of phosphatidylinositol-4,5-bisphosphate (PIP₂) to generate phosphatidylinositol-3,4,5-triphosphate (PIP₃), a second messenger involved in the phosphorylation of AKT and other components in the AKT/mTOR pathway. Up to 70% of breast cancers have some form of molecular aberration of the PI3K/AKT/mTOR pathway. Activating mutations in PIK3CA, encoding the p110α subunit of PI3K, are highly prevalent in breast cancer and solid tumor malignancies.

There is a need for active agents for treatment of Hormone Receptor Positive and HER2 Negative (HR+/HER2−) locally advanced or metastatic breast cancer.

### SUMMARY OF THE INVENTION

The present disclosure provides a combination therapy comprising a PI3K inhibitor (e.g., GDC-0077), a CDK4/6 inhibitor (e.g., palbociclib, ribociclib, or abemaciclib), and an endocrine therapy (e.g., fulvestrant or letrozole) for the treatment of breast cancers.

One aspect of the present disclosure provides a combination therapy comprising a PI3K inhibitor (e.g., GDC-0077), a CDK4/6 inhibitor (e.g., palbociclib, ribociclib, or abemaciclib), and fulvestrant for the treatment of breast cancers.

Another aspect of the present disclosure provides a combination therapy comprising a PI3K inhibitor (e.g., GDC-0077), a CDK4/6 inhibitor (e.g., palbociclib, ribociclib, or abemaciclib), and letrozole for the treatment of breast cancers.

The disclosure further provides methods of treating Hormone Receptor Positive and HER2 Negative (HR+/HER2−) locally advanced or metastatic breast cancer in a patient comprising administering a therapeutically effective amount of GDC-0077, or a pharmaceutically acceptable salt thereof, a CDK4/6 inhibitor (e.g., palbociclib, ribociclib, or abemaciclib), and an endocrine therapy (e.g., fulvestrant or letrozole).

In one aspect, the present disclosure provides a method of treating Hormone Receptor Positive and HER2 Negative (HR+/HER2−) locally advanced or metastatic breast cancer in a patient comprising administering a therapeutically effective amount of GDC-0077, or a pharmaceutically acceptable salt thereof, palbociclib and fulvestrant.

In another aspect, the present disclosure provides a method of treating Hormone Receptor Positive and HER2 Negative (HR+/HER2−) locally advanced or metastatic breast cancer in a patient comprising administering a therapeutically effective amount of GDC-0077, or a pharmaceutically acceptable salt thereof, palbociclib and letrozole.

In some embodiments, patient has PIK3CA mutant, hormone receptor-positive, Her2-negative, locally advanced or metastatic breast cancer.

Also provided is a combination for use in treating Hormone Receptor Positive and HER2 Negative (HR+/HER2−) locally advanced or metastatic breast cancer wherein said combination comprises GDC-0077, or a pharmaceutically acceptable salt thereof, a CDK4/6 inhibitor (e.g., palbociclib, ribociclib, or abemaciclib), and an endocrine therapy (e.g., fulvestrant or letrozole).

Also provided is a use of a combination in the manufacture of a medicament for treating Hormone Receptor Positive and HER2 Negative (HR+/HER2−) locally advanced or metastatic breast cancer wherein said combination comprises GDC-0077, or a pharmaceutically acceptable salt thereof, a CDK4/6 inhibitor (e.g., palbociclib, ribociclib, or abemaciclib), and an endocrine therapy (e.g., fulvestrant or letrozole).

In some embodiments, the hormone receptor-positive, Her2-negative, locally advanced or metastatic breast cancer is PIK3CA mutant or has one or more PIK3CA mutations.

In some embodiments, GDC-0077 is adminstered at a 9 mg daily dose.

In some embodiments, provided is a method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising GDC-0077, palbociclib and fulvestrant, wherein said combination therapy is administered over a 28-day cycle.

In some embodiments, provided is a method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising a dosing regimen comprising:
a. administering GDC-0077 QD on days 1-28 of a first 28-day cycle;
b. administering palbociclib QD on days 1-21 of a first 28-day cycle; and
c. administering fulvestrant on days 1 and 15 of a first 28-day cycle.

In some of these embodiments, the method further comprises one or more additional 28-day cycles comprising:
a. administering GDC-0077 on days 1-28 of each additional 28-day cycle;
b. administering palbociclib on days 1-21 of each additional 28-day cycle; and
c. administering fulvestrant on day 1 of each additional 28-day cycle (or approximately once every 4 weeks).

Also provided is a combination for use in treating a PIK3CA mutant, hormone receptor positive and HER2 negative locally advanced or metastatic breast cancer, wherein said combination comprises GDC-0077, palbociclib and fulvestrant, and wherein said combination is administered over a 28-day cycle.

Also provided is a combination for use in treating a PIK3CA mutant, hormone receptor positive and HER2 negative locally advanced or metastatic breast cancer, wherein said combination is administered in a combination therapy comprising a dosing regimen comprising:
a. administering GDC-0077 QD on days 1-28 of a first 28-day cycle;
b. administering palbociclib QD on days 1-21 of a first 28-day cycle; and
c. administering fulvestrant on days 1 and 15 of a first 28-day cycle.

Also provided is a use of a combination in the manufacture of a medicament for treating a PIK3CA mutant, hormone receptor positive and HER2 negative locally advanced or metastatic breast cancer, wherein said combination comprises GDC-0077, palbociclib and fulvestrant, and wherein said combination is administered over a 28-day cycle.

Also provided is a use of a combination in the manufacture of a medicament for treating a PIK3CA mutant, hormone receptor positive and HER2 negative locally advanced or metastatic breast cancer, wherein said combination is administered in a combination therapy comprising a dosing regimen comprising:
a. administering GDC-0077 QD on days 1-28 of a first 28-day cycle;
b. administering palbociclib QD on days 1-21 of a first 28-day cycle; and
c. administering fulvestrant on days 1 and 15 of a first 28-day cycle.

In some of these embodiments, the dosing regimen further comprises one or more additional 28-day cycles comprising:
a. administering GDC-0077 on days 1-28 of each additional 28-day cycle;
b. administering palbociclib on days 1-21 of each additional 28-day cycle; and
c. administering fulvestrant on day 1 of each additional 28-day cycle (or approximately once every 4 weeks).

In some of these embodiments, GDC-0077 is administered at an amount of 9 mg, e.g., in an oral tablet. In some embodiments, palbociclib is administered at an amount of 125 mg, e.g., in an oral capsule or tablet. In some embodiments, fulvestrant is administered at an amount of 500 mg, e.g., by intramuscular (IM) injection or infusion.

In some embodiments, provided is a method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising GDC-0077, palbociclib and letrozole, wherein said combination therapy is administered over a 28-day cycle.

In some embodiments, provided is a method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising a dosing regimen comprising:
a. administering GDC-0077 QD on days 1-28 of a first 28-day cycle;
b. administering palbociclib QD on days 1-21 of a first 28-day cycle; and
c. administering letrozole QD on days 1-28 of a first 28-day cycle.

In some of these embodiments, the method further comprises one or more additional 28-day cycles comprising:
a. administering GDC-0077 on days 1-28 of each additional 28-day cycle;
b. administering palbociclib on days 1-21 of each additional 28-day cycle; and
c. administering letrozole on days 1-28 of each additional 28-day cycle.

Also provided is a combination for use in treating a PIK3CA mutant, hormone receptor positive and HER2 negative locally advanced or metastatic breast cancer, wherein said combination comprises GDC-0077, palbociclib and letrozole, and wherein said combination is administered over a 28-day cycle.

Also provided is a combination for use in treating a PIK3CA mutant, hormone receptor positive and HER2 negative locally advanced or metastatic breast cancer, wherein said combination is administered in a combination therapy comprising a dosing regimen comprising:
a. administering GDC-0077 QD on days 1-28 of a first 28-day cycle;
b. administering palbociclib QD on days 1-21 of a first 28-day cycle; and
c. administering letrozole QD on days 1-28 of a first 28-day cycle.

Also provided is a use of a combination in the manufacture of a medicament for treating a PIK3CA mutant, hormone receptor positive and HER2 negative locally advanced or metastatic breast cancer, wherein said combination comprises GDC-0077, palbociclib and letrozole, and wherein said combination is administered over a 28-day cycle.

Also provided is a use of a combination in the manufacture of a medicament for treating a PIK3CA mutant, hormone receptor positive and HER2 negative locally advanced or metastatic breast cancer, wherein said combination is administered in a combination therapy comprising a dosing regimen comprising:
a. administering GDC-0077 QD on days 1-28 of a first 28-day cycle;
b. administering palbociclib QD on days 1-21 of a first 28-day cycle; and
c. administering letrozole QD on days 1-28 of a first 28-day cycle.

In some of these embodiments, the dosing regimen further comprises one or more additional 28-day cycles comprising:
a. administering GDC-0077 on days 1-28 of each additional 28-day cycle;
b. administering palbociclib on days 1-21 of each additional 28-day cycle; and
c. administering letrozole on days 1-28 of each additional 28-day cycle.

In some of these embodiments, GDC-0077 is administered at an amount of 3, 6 or 9 mg, e.g., in one or more oral tablets. In some embodiments, GDC-0077 is administered at an amount of 9 mg, e.g., in an oral tablet. In some embodiments, palbociclib is administered at an amount of 125 mg, e.g., in an oral capsule or tablet. In some embodiments, letrozole is administered at an amount of 2.5 mg. e.g., in an oral tablet.

In a further aspect, provided is a method of inhibiting tumor growth or producing/increasing tumor regression in a patient having PIK3CA mutant, hormone receptor positive and HER2 negative locally advanced or metastatic breast cancer, the method comprising administering to the patient a combination therapy according to the methods detailed herein.

In another aspect, provided is a combination for use in inhibiting tumor growth or producing/increasing tumor regression in a patient having PIK3CA mutant, hormone receptor positive and HER2 negative locally advanced or metastatic breast cancer, according to the combination for use detailed herein.

In another aspect, provided is a use of a combination in the manufacture of a medicament for inhibiting tumor growth or producing/increasing tumor regression in a patient having PIK3CA mutant, hormone receptor positive and HER2 negative locally advanced or metastatic breast cancer, according to the combinations or uses detailed herein.

In some of these embodiments, the patient has locally advanced or metastatic breast cancer not amenable to curative therapy. In some embodiments, the patient has progression of disease during adjuvant endocrine treatment or within 12 months of completing adjuvant endocrine therapy (e.g., with an aromatase inhibitor or tamoxifen). In some embodiments, he patient has adequate hematologic and organ function within 14 days prior to initiation of study treatment. In some embodiments, the patient is post-menopausal (e.g., a post-menopausal women). In some embodiments, the patient is premenopausal or perimenopausal (e.g., a premenopausal or a perimenopausal women). In some embodiments, the patient is male.

Further provided is a method of preventing or delaying development of resistance of a tumor (e.g., breast cancer) to a therapy containing palbociclib, comprising administering a combination therapy comprising GDC-0077, palbociclib and fulvestrant, or a combination therapy comprises GDC-0077, palbociclib and letrozole. In some embodiments, the combination therapy is administered according to any methods as detailed herein.

Also provided is a combination for use in preventing or delaying development of resistance of a tumor (e.g., breast cancer) to a therapy containing palbociclib, wherein said combination comprises GDC-0077, palbociclib and fulvestrant, or comprises GDC-0077, palbociclib and letrozole. In some embodiments, said combination is administered according to any uses as detailed herein.

Also provided is a use of a combination in the manufacture of a medicament for preventing or delaying development of resistance of a tumor (e.g., breast cancer) to a therapy containing palbociclib, wherein said combination comprises GDC-0077, palbociclib and fulvestrant, or comprises GDC-0077, palbociclib and letrozole. In some embodiments, said combination is administered according to any uses as detailed herein.

### BRIEF DESCRIPTION OF THE DRAWINGS

FIG. 1 shows the effects of each single agent (P, F, and G), double combinations (P+F, G+F, and G+P) and the triple combination (G+P+F) in a MCF-7 PIK3CA-mutant E545K ER+ breast carcinoma xenograft mouse model where each mouse (N=12) received 25 mg/kg (oral, QD) GDC-0077 (G) for 21 days, 50 mg/kg (oral, QD) palbociclib (P) for 21 days, and 200 mg/kg (s.c., weekly) fulvestrant (F) for 3 weeks.

FIG. 2 shows the body weigh change in a MCF-7 PIK3CA-mutant E545K ER+breast carcinoma xenograft mouse model where each mouse received 50 mg/kg (oral, QD) GDC-0077 (G) for 21 days, 50 mg/kg (oral, QD) palbociclib (P) for 21 days, and 200 mg/kg (s.c., weekly) fulvestrant (F) for 3 weeks.

FIG. 3 shows GDC-0077 single agent antitumor activity in a Phase 1a clinical study.

FIG. 4 shows anti-tumor activity of GDC-0077 (G) in combination with letrozole (L). Footnotes: * p110α mutation: KIN=kinase domain (H1047, M1043); HEL=helical domain (E545, E542, Q546); Mul=multiple mutations; O=other: N345K. ** Prior AI=prior aromatase inhibitor; A=adjuvant; M=metastatic setting, B=both adjuvant and metastatic. Shaded squares in Time on Treatment row indicate >6 months on treatment.

FIG. 5 shows anti-tumor activity of GDC-0077 (G) in combination with palbociclib (P) and letrozole (L). Footnotes: * p110α mutation: kinase domain (H1047, M1043); HEL=helical domain (E545, E542, Q546); Mul=multiple mutations. ** Prior AI=prior aromatase inhibitor; A=adjuvant; M=metastatic setting; B=both adjuvant and metastatic. *** Not CR since patients still had non-measurable disease. Shaded squares in Time on Treatment row indicate >6 months on treatment.

FIG. 6 shows anti-tumor activity of GDC-0077 (G) in combination with Fulvestrant (F).

FIG. 7 shows anti-tumor activity of inavolisib in combination with palbociclib and fulvestrant in Arm E. Footnotes: * p110α mutation: HEL, helical (E545, E542, Q546); KIN, kinase (H1047, M1043); MUL, multiple mutations. Mutations were defined using local and/or central Cobas® testing. t Prior AI, prior aromatase inhibitor; A, adjuvant; M, metastatic setting, B, both adjuvant and metastatic. AI, aromatase inhibitor; CR, complete response; PD, disease progression; PR, partial response; SD, stable disease; SLD, sum of longest diameters. Shaded squares in Time on Treatment row indicate >6 months on treatment.

FIG. 8 shows anti-tumor activity of inavolisib in combination with palbociclib and fulvestrant in Arm F. Footnotes: * p110α mutation: HEL, helical (E545, E542, Q546); KIN, kinase (H1047, M1043); MUL, multiple mutations. Mutations were defined using local and/or central Cobas® testing. t Prior AI, prior aromatase inhibitor; A, adjuvant; M, metastatic setting, B, both adjuvant and metastatic. AI, aromatase inhibitor; CR, complete response; PD, disease progression; PR, partial response; SD, stable disease; SLD, sum of longest diameters. Shaded squares in Time on Treatment row indicate >6 months on treatment.

### DETAILED DESCRIPTION OF THE INVENTION

### Definitions

The words “comprise,” “comprising,” “include,” “including,” and “includes” when used in this specification and claims are intended to specify the presence of stated features, integers, components, or steps, but they do not preclude the presence or addition of one or more other features, integers, components, steps, or groups thereof.

The terms “treat” and “treatment” refer to both therapeutic treatment and prophylactic or preventative measures, wherein the object is to prevent or slow down (lessen) an undesired physiological change or disorder, such as the growth, development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

“Time to progression” or “TTP” refers to the time from randomization until objective tumor progression.

“Objective response rate” or “ORR” refers to the proportion of patients with a confirmed complete response or partial response on two consecutive occasions ≥4 weeks apart, as determined by the investigator according to RECIST v1.1

“Best overall response rate” or “BOR” refers to the proportion of patients with a CR or PR, as determined by the investigator according to RECIST v1.1

“Duration of response” or “DOR” refers to the time from the first occurrence of a documented objective response to disease progression, as determined by the investigator according to RECIST v1.1, or death from any cause, whichever occurs first.

“Clinical benefit rate” or “CBR” refers to the proportion of patients with stable disease for at least 24 weeks or with confirmed complete or partial response, as determined by the investigator according to RECIST v1.1.

“Overall survival” or “OS” refers to the time from enrollment to death from any cause.

“Time to deterioration (TTD) in pain” refers to the time from randomization to the first documentation of a ≥2-point increase from baseline on the “worst pain” item from the Brief Pain Inventory-Short Form (BPI-SF).

“Time to deterioration (TTD) in Physical Function” refers to the time from randomization to the first documentation of a ≥10-point decrease from baseline in the European Organisation for Research and Treatment of Cancer Quality of Life-Core 30 Questionnaire (EORTC QLQ-C30) Physical Function scale (items 1-5).

“Time to deterioration (TTD) in Role Function” refers to the time from randomization to the first documentation of a ≥10-point decrease from baseline in the EORTC QLQ-C30 Role Function scale (items 6 and 7).

“Time to deterioration (TTD) in global health status (GHS)/health-related quality of life (HRQoL)” refers to the time from randomization to the first documentation of a ≥10-point decrease from baseline in the EORTC QLQ-30 GHS/HRQoL scale (items 29 and 30).

“Progression free survival” or “PFS” refers to the time from enrollment to the date of the first recorded occurrence of disease progression, as determined by the investigator using RECIST v1.1 or death from any cause, whichever occurs first.

“Complete response” or “CR” refers to the disappearance of all target lesions and non-target lesions and (if applicable) normalization of tumor marker level.

“Partial response”, “PR” or “Non-CR/Non-PrD” refers to persistence of one or more non-target lesions and/or (if applicable) maintenance of tumor marker level above the normal limits. A PR can also refer to ≥30% decrease in sum of diameters of target lesions, in the absence of CR, new lesions, and unequivocal progression in non-target lesions.

“Progressive disease” or “PrD” refers to ≥20% increase in sum of diameters of target lesions, unequivocal progression in non-target lesions, and/or appearance of new lesions.

“Stable disease” or “SD” refers to neither sufficient shrinkage to qualify for CR or PR nor sufficient increase growth of tumor to qualify for PrD.

An “administration period” or “cycle” refers to a period of time comprising administration of one or more agents described herein and an optional period of time comprising no administration of one or more of the agents described herein. For example, a cycle can be 28 days in total length and include administration of one or more agents for 21 days and a rest period of 7 days. A “rest period” refers to a period of time where at least one of the agents described herein are not administered. In one embodiment, a rest period refers to a period of time where none of the agents described herein are administered.

A “dosing regimen” refers to a period of administration of the agents described herein comprising one or more cycles, where each cycle can include administration of the agents described herein at different times or in different amounts.

“QD” refers to administration of a compound once daily.

A graded adverse event refers to the severity grading scale as established for by NCI CTCAE. In one embodiment, the adverse event is graded in accordance with the table below.

| Grade | Severity |
|---|---|
| 1 | Mild; asymptomatic or mild symptoms; clinical or diagnostic |
| | observations only; or intervention not indicated |
| 2 | Moderate; minimal, local, or non-invasive intervention indicated; |
| | or limiting age-appropriate instrumental activities of daily living |
| 3 | Severe or medically significant, but not immediately life- |
| | threatening; hospitalization or prolongation of hospitalization |
| | indicated; disabling; or limiting self-care activities of daily living |
| 4 | Life-threatening consequences or urgent intervention indicated |
| 5 | Death related to adverse event |

The term “detection” includes any means of detecting, including direct and indirect detection.

The term “prognosis” is used herein to refer to the prediction of the likelihood of cancer-attributable death or progression, including, for example, recurrence, metastatic spread, and drug resistance, of a neoplastic disease, such as cancer.

The term “prediction” (and variations such as predicting) is used herein to refer to the likelihood that a patient will respond either favorably or unfavorably to a drug or set of drugs. In one embodiment, the prediction relates to the extent of those responses. In another embodiment, the prediction relates to whether and/or the probability that a patient will survive following treatment, for example treatment with a particular therapeutic agent and/or surgical removal of the primary tumor, and/or chemotherapy for a certain period of time without cancer recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, chemotherapy, etc., or whether long-term survival of the patient, following a therapeutic regimen is likely.

The term “increased resistance” to a particular therapeutic agent or treatment option, when used in accordance with the invention, means decreased response to a standard dose of the drug or to a standard treatment protocol.

“Response” can be assessed using any endpoint indicating a benefit to the patient, including, without limitation, (1) inhibition, to some extent, of tumor growth, including slowing down or complete growth arrest; (2) reduction in the number of tumor cells; (3) reduction in tumor size; (4) inhibition (e.g., reduction, slowing down or complete stopping) of tumor cell infiltration into adjacent peripheral organs and/or tissues; (5) inhibition (e.g., reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

A “biomarker” is a characteristic that is objectively measured and evaluated as an indicator of normal biological processes, pathogenic processes, or pharmacological responses to a therapeutic intervention. Biomarkers may be of several types: predictive, prognostic, or pharmacodynamics (PD). Predictive biomarkers predict which patients are likely to respond or benefit from a particular therapy. Prognostic biomarkers predict the likely course of the patient's disease and may guide treatment. Pharmacodynamic biomarkers confirm drug activity, and enables optimization of dose and administration schedule.

“Change” or “modulation” of the status of a biomarker, including a PIK3CA mutation or set of PIK3CA mutations, as it occurs in vitro or in vivo is detected by analysis of a biological sample using one or more methods commonly employed in establishing pharmacodynamics (PD), including: (1) sequencing the genomic DNA or reverse-transcribed PCR products of the biological sample, whereby one or more mutations are detected; (2) evaluating gene expression levels by quantitation of message level or assessment of copy number; and (3) analysis of proteins by immunohistochemistry (IHC), immunocytochemistry, ELISA, or mass spectrometry whereby degradation, stabilization, or post-translational modifications of the proteins such as phosphorylation or ubiquitination is detected.

A “chemotherapeutic agent” is a biological (large molecule) or chemical (small molecule) compound useful in the treatment of cancer, regardless of mechanism of action.

The term “package insert” is used to refer to instructions customarily included in commercial packages of therapeutic products, that contain information about the indications, usage, dosage, administration, contraindications and/or warnings concerning the use of such therapeutic products.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

The desired pharmaceutically acceptable salt may be prepared by any suitable method available in the art. For example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. Acids which are generally considered suitable for the formation of pharmaceutically useful or acceptable salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1 19; P. Gould, International J. of Pharmaceutics (1986) 33 201 217; Anderson et al, The Practice of Medicinal Chemistry (1996), Academic Press, New York; Remington's Pharmaceutical Sciences, 18^{th} ed., (1995) Mack Publishing Co., Easton PA; and in The Orange Book (Food & Drug Administration, Washington, D.C. on their website). These disclosures are incorporated herein by reference thereto.

The phrase “pharmaceutically acceptable” indicates that the substance or composition must be compatible chemically and/or toxicologically, with the other ingredients comprising a formulation, and/or the patient being treated therewith.

The term “synergistic” as used herein refers to a therapeutic combination which is more effective than the additive effects of the two or more single agents. A determination of a synergistic interaction between a compound of GDC-0077 or a pharmaceutically acceptable salt thereof, and one or more chemotherapeutic agent may be based on the results obtained from the assays described herein. The results of these assays can be analyzed using the Chou and Talalay combination method and Dose-Effect Analysis with CalcuSyn® software in order to obtain a Combination Index (Chou and Talalay, 1984, Adv. Enzyme Regul. 22:27-55). The combinations provided by this invention have been evaluated in several assay systems, and the data can be analyzed utilizing a standard program for quantifying synergism, additivism, and antagonism among anticancer agents described by Chou and Talalay, in “New Avenues in Developmental Cancer Chemotherapy,” Academic Press, 1987, Chapter 2. Combination Index values less than 0.8 indicates synergy, values greater than 1.2 indicate antagonism and values between 0.8 and 1.2 indicate additive effects. The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes or in separate pills or tablets. In general, during alternation therapy, an effective dosage of each active ingredient is administered sequentially, i.e., serially, whereas in combination therapy, effective dosages of two or more active ingredients are administered together. Combination effects were evaluated using both the BLISS independence model and the highest single agent (HSA) model (Lehir et al. 2007, Molecular Systems Biology 3:80). BLISS scores quantify degree of potentiation from single agents and a BLISS score >0 suggests greater than simple additivity. An HSA score >0 suggests a combination effect greater than the maximum of the single agent responses at corresponding concentrations.

### Clinical Compounds

Inavolisib (a.k.a. GDC-0077):

Inavolisib (GDC-0077) is a potent, orally bioavailable, clinical-stage, selective inhibitor of the Class I PI3K alpha isoform (PI3Ku), with >300-fold less potent biochemical inhibition for other Class I PI3K beta, delta, and gamma isoforms and increased potency in tumor cells bearing mutant PI3K over wild type (WT) PI3K cells (Braun, M. et al “Discovery of GDC-0077: A highly selective inhibitor of PI3K-alpha that induces degradation of mutant-p110 alpha protein” Abstracts of Papers, 254th ACS National Meeting & Exposition, Washington, DC, USA, Aug. 20-24, 2017, MEDI-22; Garland, K. et al “Discovery of novel class of alpha selective PI3K inhibitors” Abstracts of Papers, 254th ACS National Meeting & Exposition, Washington, DC, USA, Aug. 20-24, 2017, MEDI-103; Hong, R. et al “GDC-0077 is a selective PI3K alpha inhibitor that demonstrates robust efficacy in PIK3CA mutant breast cancer models as a single agent and in combination with standard of care therapies” 2017 San Antonio Breast Cancer Symposium, Dec. 5-9 2017, San Antonio, TX, Abstract Publication Number: PD4-14; Edgar, K. et al “Preclinical characterization of GDC-0077, a specific PI3K alpha inhibitor in early clinical development” Cancer Research 77(13 Supplement): Abstract 156 July 2017).

Inavolisib, CAS Registry Number 2060571-02-8, Genentech, Inc., U.S. Pat. No. 9,650,393; named as (S)-2-((2-((S)-4-(difluoromethyl)-2-oxooxazolidin-3-yl)-5,6-dihydrobenzo[f]imidazo[1,2-d][1,4]oxazepin-9-yl)amino)propanamide, has the structure:

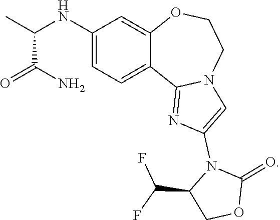 [SMILES: C[C@H](Nc1ccc2c(c1)OCCn1cc(N3C(=O)OC[C@H]3C(F)F)nc1-2)C(N)=O]

Inavolisib is also known as GDC-0077, RG6114, RO7113755, or chemical name (2S)-2-[[2-[(4S)-4-(Difluoromethyl)-2-oxo-3-oxazolidinyl]-5,6-dihydroimidazo[1,2-d][1,4]benzoxazepin-9-yl]amino]propanamide.

GDC-0077 exerts its activity by binding to the ATP binding site of PI3K, thereby inhibiting the phosphorylation of membrane-bound 4,5-phosphatidylinositol bisphosphate (PIP₂) to 3,4,5-phosphatidylinositol triphosphate (PIP₃). Inhibiting the phosphorylation of PIP₂ to PIP₃ decreases downstream activation of AKT and pS6, resulting in decreased cellular proliferation, metabolism, and angiogenesis. Nonclinical studies demonstrate that GDC-0077 specifically degrades mutant p110 alpha, inhibits proliferation and induces apoptosis of PIK3CA-mutant breast cancer cell lines, inhibits tumor growth in human breast xenograft models harboring PIK3CA mutations, and reduces downstream PI3K-pathway markers, including pAKT (phosphorylated form of AKT), pPRAS40, and pS6.

### Palbociclib:

Palbociclib is a selective inhibitor of the cyclin-dependent kinases CDK4 and CDK6 (Finn et al (2009) Breast cancer research: BCR 11 (5):R77; Rocca et al (2014) Expert Opin Pharmacother 15 (3):407-20; U.S. Pat. Nos. 6,936,612; 7,863,278; 7,208,489; 7,456,168). Palbociclib can be prepared and characterized as described in U.S. Pat. No. 7,345,171. IBRANCE® is approved for the treatment of breast cancer.

Palbociclib (PD-0332991, IBRANCE®, Pfizer, Inc., CAS Reg. No. 571190-30-2), named as 6-acetyl-8-cyclopentyl-5-methyl-2-(5-(piperazin-1-yl)pyridin-2-ylamino)pyrido[2,3-d]pyrimidin-7(8H)-one, has the structure:

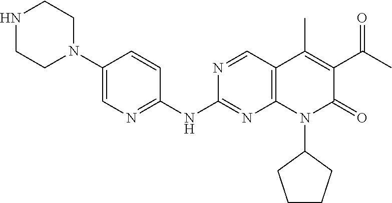 [SMILES: CC(=O)c1c(C)c2cnc(Nc3ccc(N4CCNCC4)cn3)nc2n(C2CCCC2)c1=O]

Palbociclib is a CDK4/6 inhibitor and, in combination with letrozole or fulvestrant, an effective treatment for postmenopausal patients with HR+(positive)/HER2− (negative) breast cancer. In combination with letrozole or fulvestrant, the main toxicity of palbociclib is neutropenia (Finn et al (2015) Lancet Oncol 16:25-35; Turner et al (2015) N Engl J Med 373:209-19). In combination with letrozole, 36% of patients required ≥1 dose reduction of palbociclib; dose holds and cycle delays were reported in 70% and 68% of patients, respectively (Finn et al (2016) J Clin Oncol 34(suppl; abstr 507)). In combination with fulvestrant, 34% of patients required ≥1 dose reduction of palbociclib; dose holds and cycle delays were reported in 54% and 36% of patients, respectively (Cristofanilli et al. (2016) Lancet Oncol 17:425-39). Myelosuppression is a potential toxicity of GDC-0077.

Other exemplary CDK4/6 inhibitors include, but are not limited to: ribociclib (Butanedioic acid-7-cyclopentyl-N,N-dimethyl-2-{[5-(piperazin-1-yl) pyridin-2-yl]amino}-7H-pyrrolo[2,3-d]pyrimidine-6-carboxamide (1/1); marketed as KISQALI®); abemaciclib, (2-Pyrimidinamine, N-[5-[(4-ethyl-1-piperazinyl)methyl]-2-pyridinyl]-5-fluoro-4-[4-fluoro-2-methyl-1-(1-methylethyl)-1H-benzimidazol-6-yl], marketed as VERZENIO®); and trilaciclib (2′-((5-(4-methylpiperazin-1-yl)pyridin-2-yl)amino)-7′,8′-dihydro-6′H-spiro(cyclohexane-1,9′-pyrazino(1′,2′:1,5)pyrrolo(2,3-d)pyrimidin)-6′-one).

### Fulvestrant:

Fulvestrant is an ER antagonist and an effective treatment for postmenopausal patients with HR+breast cancer that is relatively well tolerated. The expected toxicities for GDC-0077 and fulvestrant are not overlapping. It is important to test GDC-0077 in combination with both letrozole and fulvestrant, as these endocrine therapies have different mechanisms of action, different PK properties, and different potential for drug-drug interactions (DDIs) with GDC-0077.

Fulvestrant (FASLODEX®, AstraZeneca, CAS Reg. No. 129453-61-8) is approved by the FDA for treatment of hormone receptor-positive (HR+) metastatic breast cancer in postmenopausal women with disease progression following anti-estrogen therapy (Kansra (2005) Mol Cell Endocrinol 239(1-2):27-36; Flemming et al (2009) Breast Cancer Res Treat. May; 115(2):255-68; Valachis et al (2010) Crit Rev Oncol Hematol. March; 73(3):220-7). Fulvestrant is an estrogen receptor (ER) antagonist with no agonist effects, which works both by down-regulating and by degrading the estrogen receptor (Croxtall (2011) Drugs 71(3):363-380). Fulvestrant is also a selective estrogen receptor down-regulator (SERD).

Fulvestrant is named as (7α,17β)-7-{9-[(4,4,5,5,5-pentafluoropentyl)sulfinyl]nonyl}estra-1,3,5(10)-triene-3,17-diol and has the structure:

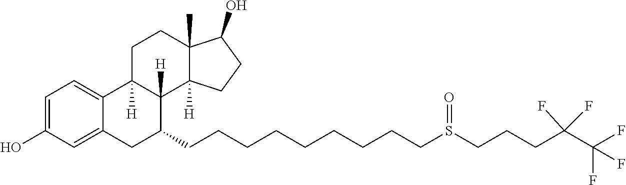

Fulvestrant belongs to a class of reversible steroidal ER antagonists that directly competes with estrogen for ER binding and is devoid of the partial agonist properties of tamoxifen. Upon binding to ER, it blocks estrogen signaling and increases the degradation of ER protein. The affinity of fulvestrant for the ER is approximately 100-fold greater than that of tamoxifen (Howell et al. (2000) Cancer 89:817-25). Fulvestrant (250 mg once monthly) was approved by the FDA in 2002 and by the EMA in 2004 for the treatment of HR-positive MBC in postmenopausal women with disease progression following anti-estrogen therapy. In multicenter Phase III studies, fulvestrant was found to be at least equivalent to anastrozole (a non-steroidal AI) in the second-line setting (Howell et al. (2002) J Clin Oncol 20:3396-3403; Osborne C K, et al (2002) J Clin Oncol 20:3386-95). Fulvestrant is also as active as tamoxifen for the first-line treatment of advanced breast cancer (Howell et al. (2004) J Clin Oncol 22:1605-1613) and displays a level of activity in patients in the post-AI metastatic disease setting similar to that of the non-steroidal AI exemestane (Chia et al. (2008) J Clin Oncol 26:1664-1670). High-dose fulvestrant (500 mg once monthly) has been demonstrated to be at least as effective as anastrozole in terms of clinical benefit rate (CBR) and overall response rate and to be associated with significantly longer time to progression for the first-line treatment of women with advanced HR-positive breast cancer (Robertson et al. (2009) J Clin Oncol 27:4530-4535). High-dose fulvestrant recently demonstrated superior progression-free survival (PFS) in women with ER-positive advanced breast cancer treated with 500 mg versus patients treated with 250 mg (Di Leo et al. (2010) J Clin Oncol 28:4594-4600). Fulvestrant (250 mg and 500 mg) was well tolerated in these studies and produced fewer estrogenic effects than did tamoxifen and resulted in less arthralgia than did the AI anastrozole (Osborne et al. (2002) J Clin Oncol 20:3386-3395). These results led to the approval of 500 mg fulvestrant given once a month as the currently approved recommended dose in the United States and the European Union (in 2010) for postmenopausal women whose disease has spread after treatment with an AI. These studies demonstrate that fulvestrant is an important treatment option for patients with advanced breast cancer and, as such, is considered appropriate control therapy for the present study.

### Letrozole:

Letrozole is an effective treatment for postmenopausal patients with HR+breast cancer that is relatively well tolerated. The expected toxicities for GDC-0077 and letrozole are not overlapping. Letrozole (FEMARA®, Novartis Pharm.) is an oral non-steroidal aromatase inhibitor for the treatment of hormonally-responsive breast cancer after surgery (Bhatnagar et al (1990) J. Steroid Biochem. and Mol. Biol. 37:1021; Lipton et al (1995) Cancer 75:2132; Goss, P. E. and Smith, R. E. (2002) Expert Rev. Anticancer Ther. 2:249-260; Lang et al (1993) The Journal of Steroid Biochem. and Mol. Biol. 44 (4-6):421-8; EP 236940; U.S. Pat. No. 4,978,672). FEMARA® is approved by the FDA for the treatment of local or metastatic breast cancer that is hormone receptor positive (HR+) or has an unknown receptor status in postmenopausal women.

Letrozole is named as 4,4′-((1H-1,2,4-triazol-1-yl)methylene)dibenzonitrile (CAS Reg. No. 112809-51-5), and has the structure:

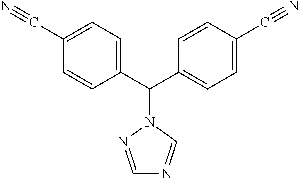 [SMILES: N#Cc1ccc(C(c2ccc(C#N)cc2)n2cncn2)cc1]

### Combination Therapy

Provided herein are combinations or combination therapies comprising a PI3K inhibitor (e.g., GDC-0077), a CDK4/6 inhibitor (e.g., palbociclib, ribociclib, or abemaciclib), and an endocrine therapy (e.g., fulvestrant or letrozole). In one aspect, provided is a combination or combination therapy comprising a PI3K alpha (PI3Ku) inhibitor (e.g., GDC-0077), a CDK4/6 inhibitor (e.g., palbociclib, ribociclib, or abemaciclib), and an endocrine therapy (e.g., fulvestrant or letrozole). In one embodiment, the combination or combination therapy comprises GDC-0077, palbociclib and fulvestrant. In another embodiment, the combination or combination therapy comprises GDC-0077, palbociclib and letrozole.

The combinations or combination therapies described herein can be provided as a kit comprising one or more of the agents for administration. In one embodiment, the kit includes GDC-0077 and fulvestrant. In one embodiment, the kit includes GDC-0077, palbociclib and letrozole. In another embodiment, the kit includes GDC-0077, palbociclib and fulvestrant. In one embodiment, the agents of the combination or combination therapy described herein are supplied in a kit in a form ready for administration or, for example, for reconstitution. Kits described herein can include instructions such as package inserts. In one embodiment, the instructions are package inserts—one for each agent in the kit.

Further provided are kits for carrying out the methods detailed herein, which comprises a pharmaceutical composition or a combination therapy described herein and instructions for use in the treatment of breast cancer.

Kits generally comprise suitable packaging. The kits may comprise one or more containers comprising any pharmaceutical composition described herein. Each component (if there is more than one component) can be packaged in separate containers or some components can be combined in one container where cross-reactivity and shelf life permit. One or more components of a kit may be sterile and/or may be contained within sterile packaging.

### Methods

Provided herein are methods of treating hormone receptor positive and HER2 negative locally advanced or metastatic breast cancer. In one embodiment, the method comprises treating hormone receptor positive and HER2 negative locally advanced or metastatic breast cancer in a patient having PIK3CA mutant, hormone receptor positive and HER2 negative locally advanced or metastatic breast cancer, by administering to the patient a combination therapy that includes a PI3K inhibitor (preferably a PI3Ku inhibitor, e.g., GDC-0077), a CDK4/6 inhibitor (e.g., palbociclib, ribociclib, or abemaciclib), and an endocrine therapy (e.g., fulvestrant or letrozole).

Also provided are methods of treating hormone receptor positive and HER2 negative (HR+/HER2−) locally advanced or metastatic breast cancer in a patient comprising administering a therapeutically effective amount of GDC-0077, or a pharmaceutically acceptable salt thereof, a CDK4/6 inhibitor (e.g., palbociclib, ribociclib, or abemaciclib), and an endocrine therapy (e.g., fulvestrant or letrozole). In some embodiments, the CDK4/6 inhibitor is palbociclib. In some embodiments, the CDK4/6 inhibitor is ribociclib or abemaciclib. In some embodiments, the endocrine therapy is fulvestrant. In some embodiments, the endocrine therapy is letrozole.

In one aspect, provided is a method of treating hormone receptor positive and HER2 negative (HR+/HER2−) locally advanced or metastatic breast cancer in a patient comprising administering a therapeutically effective amount of GDC-0077, or a pharmaceutically acceptable salt thereof, palbociclib and fulvestrant.

In another aspect, the present disclosure provides a method of treating hormone receptor positive and HER2 negative (HR+/HER2−) locally advanced or metastatic breast cancer in a patient comprising administering a therapeutically effective amount of GDC-0077, or a pharmaceutically acceptable salt thereof, palbociclib and letrozole.

In some embodiments, provided is a method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising GDC-0077, palbociclib and fulvestrant, wherein said combination therapy is administered over a 28-day cycle.

In some embodiments, provided is a method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising a dosing regimen comprising:
a. administering GDC-0077 QD on days 1-28 of a first 28-day cycle;
b. administering palbociclib QD on days 1-21 of a first 28-day cycle; and
c. administering fulvestrant on days 1 and 15 of a first 28-day cycle.

In some of these embodiments, the method further comprises one or more additional 28-day cycles comprising:
a. administering GDC-0077 on days 1-28 of each additional 28-day cycle;
b. administering palbociclib on days 1-21 of each additional 28-day cycle; and
c. administering fulvestrant on day 1 of each additional 28-day cycle (or approximately once every 4 weeks).

In some embodiments, the patient has PIK3CA mutant, hormone receptor-positive, Her2-negative, locally advanced or metastatic breast cancer. In some embodiments, the patient has mutant PIK3CA having mutations at one or more of positions 88, 106, 111, 118, 345, 420, 453, 542, 545, 546, 1043, 1047 and 1049. In some embodiments, the patient has mutant PIK3CA having mutations at one or more of H1047, E545, E542, Q546, N345, C420, M1043, G1049, E453, K111, G106, G118, and R88. In some embodiments, the patient has mutant PIK3CA containing one or more mutations selected from the group consisting of H1047D/I/L/N/P/Q/R/T/Y, E545A/D/G/K/L/Q/R/V, E542A/D/G/K/Q/R/V, Q546E/H/K/L/P/R, N345D/H/I/K/S/T/Y, C420R, M1043I/T/V, G1049A/C/D/R/S, E453A/D/G/K/Q/V, K111N/R/E, G106A/D/R/S/V, G118D, and R88Q. In some embodiments, the patient has mutant PIK3CA containing one or more mutations selected from the group consisting of E542K, E545K, Q546R, H1047L and H1047R. In some embodiments, the patient has breast cancer expressing a PIK3CA mutant selected from the group consisting of H1047D/I/L/N/P/Q/R/T/Y, E545A/D/G/K/L/Q/R/V, E542A/D/G/K/Q/R/V, Q546E/H/K/L/P/R, N345D/H/I/K/S/T/Y, C420R, M1043I/TN, G1049A/C/D/R/S, E453A/D/G/K/Q/V, K111N/R/E, G106A/D/R/S/V, G118D, and R88Q. In some embodiments, the patient has breast cancer expressing a PIK3CA mutant selected from the group consisting of E542K, E545K, Q546R, H1047L and H1047R. In some embodiments, the patient has mutant PIK3CA containing one mutation selected from the group consisting of E542K, E545K, Q546R, H1047L and H1047R, and a second mutation (e.g., a second mutation selected from E453Q/K, E726K and M1043L/I). In some embodiments, the patient has breast cancer expressing a PIK3CA mutant expressing a double mutation selected from the group consisting of E542K+E453Q/K, E542K+E726K, E542K+M1043L/I; E545K+E453Q/K, E545K+E726K, E545K+M1043L/I; H1047R+E453Q/K, and H1047R+E726K.

PIK3CA-mutant tumor status can be assessed by either central testing of blood or local testing of blood or tumor tissue. In some embodiments, the central test for identification of eligible PIK3CA mutations is the FoundationOne Liquid Clinical Trial Assay performed at Foundation Medicine, Inc. In some embodiments, the local tests of blood or tumor tissue is performed using a Sponsor pre-approved PCR- or NGS-based assay at a CLIA-certified or equivalent laboratory.

In some embodiments, provided is a method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising GDC-0077, palbociclib and letrozole, wherein said combination therapy is administered over a 28-day cycle.

In some embodiments, provided is a method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising a dosing regimen comprising:
a. administering GDC-0077 QD on days 1-28 of a first 28-day cycle;
b. administering palbociclib QD on days 1-21 of a first 28-day cycle; and
c. administering letrozole QD on days 1-28 of a first 28-day cycle.

In some of these embodiments, the method further comprises one or more additional 28-day cycles comprising:
a. administering GDC-0077 on days 1-28 of each additional 28-day cycle;
b. administering palbociclib on days 1-21 of each additional 28-day cycle; and
c. administering letrozole on days 1-28 of each additional 28-day cycle.

In one embodiment, the method includes a combination therapy comprising (i) GDC-0077; (ii) fulvestrant; and (iii) palbociclib. In one embodiment, the method includes a combination therapy comprising (i) GDC-0077; (ii) palbociclib; and (iii) fulvestrant administered in accordance with a dosing regimen described herein.

In one embodiment, the method includes a combination therapy comprising (i) GDC-0077; (ii) fulvestrant; and (iii) palbociclib. In one embodiment, the method includes a combination therapy comprising (i) GDC-0077; (ii) palbociclib; and (iii) letrozole administered in accordance with a dosing regimen described herein.

In a further aspect, provided is a method of inhibiting tumor growth or producing/increasing tumor regression in a patient having PIK3CA mutant, hormone receptor positive and HER2 negative locally advanced or metastatic breast cancer, the method comprising administering to the patient a combination therapy according to the methods detailed herein.

In some embodiments, estrogen receptor (ER)-positive and/or progesterone receptor-positive tumor are documented according to American Society of Clinical Oncology/College of American Pathologists (ASCO/CAP) guidelines, defined as 1% of tumor cells stained positive based on the most recent tumor biopsy and assessed locally.

In some embodiments HER2-negative tumor are documented according to ASCO/CAP guidelines, defined as a HER2 immunohistochemistry (IHC) score of 0 or 1+, or an IHC score of 2+ accompanied by a negative fluorescence, chromogenic, or silver in situ hybridization test indicating the absence of HER2 gene amplification, or a HER2/CEP17 ratio of <2.0 based on the most recent tumor biopsy and assessed locally.

In some embodiments, the patient is women or men ≥18 years of age.

In some embodiments, the patient is postmenopausal (e.g., a postmenopausal female). Postmenopausal female is defined by at least one of the following criteria: (1) Age ≥60 years; (2) Age <60 years and 12 months of amenorrhea plus follicle-stimulating hormone and plasma estradiol levels within postmenopausal range by local laboratory assessment in the absence of oral contraceptive pills, hormone replacement therapy, or gonadotropin releasing hormone agonist or antagonist; (3) Documented bilateral oophorectomy (≥14 days prior to first treatment on Day 1 of Cycle 1 and recovery to baseline).

In some embodiments, the patient is premenopausal or perimenopausal female (i.e., not meeting the criteria for postmenopausal) and has been treated with luteinizing hormone-releasing hormone (LHRH) agonist therapy (e.g., goserelin or leuprolide) beginning at least 2 weeks prior to Day 1 of Cycle 1 and continuing for the duration of study treatment.

In some embodiments, the patient is male and has been treated with with LHRH agonist therapy (e.g., goserelin or leuprolide) beginning at least 2 weeks prior to Day 1 of Cycle 1 and continuing for the duration of study treatment.

Agents described herein can be administered in accordance with a package insert. In one embodiment of the methods described herein, agents can be administered in an effective amount as described herein. In some embodiments, palbociclib, fulvestrant or letrozole, where applicable, is administered at its approved dosage by an approved route of administration.

Agents in a combination therapy detailed herein may be administered simultaneously or sequentially. Two of the triplets of a combination therapy may be administer simultaneously while the third agent may be adminstered before or after. For example, in one embodiment of the methods described herein, administration of GDC-0077 occurs before administration of another agent (e.g. fulvestrant or letrozole). In one embodiment of the methods described herein, administration of GDC-0077 occurs before administration of fulvestrant or letrozole and the administration of fulvestrant or letrozole occurs before the administration of a CDK4/6 inhibitor (e.g. palbociclib). In another embodiment, GDC-0077 is administered prior to or concurrently with palbociclib and fulvestrant or letrozole is administered thereafter.

In some embodiments, GDC-0077 is administered at an amount of 3, 6 or 9 mg, e.g., in one or more oral tablets. In some embodiments, GDC-0077 is adminstered orally at a 9 mg daily dose. In some of these embodiments, GDC-0077 is administered at an amount of 9 mg, e.g., in an oral tablet.

In one embodiment of the methods described herein, palbociclib is administered as an agent of the triple combination therapy described herein. In one embodiment, palbociclib is administered orally at an amount of 125 mg, 100 mg, or 75 mg. In another embodiment, palbociclib is administered orally at an amount of 125 mg. In another embodiment, palbociclib is administered orally at an amount of 100 mg. In still another embodiment, palbociclib is administered orally at an amount of 75 mg. Is such embodiments, palbociclib is administered QD on days 1-21 of each 28-day cycle. In another embodiment of the methods described herein, palbociclib is administered in accordance with a package insert. In one embodiment, palbociclib is administered orally QD on days 1-21 of each 28-day cycle at an amount described herein. In still another embodiment, the amount of palbociclib is modified (e.g. reduced) from the initial dosage. In one such embodiment, the amount of palbociclib administered is reduced from 125 mg to 100 mg and can be, in one embodiment, further reduced to 75 mg. In another embodiment, palbociclib is administered in a dosing regimen as described herein.

In some embodiments, palbociclib is administered at an amount of 125 mg, e.g., in an oral capsule or tablet.

In another embodiment of the methods described herein, fulvestrant is administered at a dose of about 500 mg. In one embodiment of the methods described herein, fulvestrant is administered in accordance with a package insert. In one embodiment, fulvestrant is administered as two separate 250 mg intramuscular injections. In another embodiment, fulvestrant is administered in a dosing regimen as described herein. In one such embodiment, fulvestrant is administered on days 1 and 15 of the first 28-day cycle and on day 1 of each subsequent 28-day cycle thereafter.

In some embodiments, fulvestrant is administered at an amount of 500 mg, e.g., by intramuscular (IM) infusion.

In another embodiment of the methods described herein, letrozole is administered at a dose of about 2.5 mg. In one embodiment of the methods described herein, letrozole is administered in accordance with a package insert. In another embodiment, fulvestrant is administered in a dosing regimen as described herein. In one such embodiment, letrozole is administered orally at a dose of about 2.5 mg QD.

In some embodiments, letrozole is administered at an amount of 2.5 mg. e.g., in an oral tablet.

In one embodiment, the methods described herein include a combination therapy described herein administered according to a dosing regimen comprising one 28-day cycle.

In another embodiment, the methods described herein include a combination therapy described herein administered according to a dosing regimen comprising a first 28-day cycle followed by additional 28-day cycles. In another embodiment, the methods described herein include a combination therapy described herein administered according to a dosing regimen comprising a first 28-day cycle followed by 2-10 28-day cycles. In still another embodiment, the methods described herein include a combination therapy described herein administered according to a dosing regimen comprising a first 28-day cycle followed by 2-8 28-day cycles. In one embodiment of the methods described herein, the dosing regimen comprises a first 28-day cycle followed by 2-36, 2-30, 2-24, 2-18, 2-12, 2-10, 2-8, 2-6, or 2-4 28-day cycles.

Further embodiments of the methods of treating hormone receptor positive and HER2 negative locally advanced or metastatic breast cancer are provided herein.

In one embodiment, the efficacy of the combination is measured as a function of PFS. In one such embodiment, PFS of the patient is increased by 3, 4, 5, 6, 7, 8, 9, 10, 15, 20, or more months compared to non-treatment or SOC treatment. In one embodiment, PFS is measured for at least 64 months following the first dosage of the combination therapy described herein. In another embodiment, the efficacy is measured as a function of PFS in a biomarker positive patient set (e.g. a biomarker panel as described herein including PIK3CA) comparable to a biomarker negative patient set.

In one embodiment, treatment with a combination therapy according to the methods provided herein increases a patient's OS by 3, 4, 5, 6, 7, 8, 9, 10, 15, 20 or more months comparable to non-treatment or SOC treatment. In one embodiment, treatment with a combination therapy according to the methods provided herein increases the patient's amount of ORR. In another embodiment, efficacy of response is measured as a function of DOR comparable to non-treatment or SOC treatment. In still another embodiment, efficacy of response is measured as a function of CBR comparable to non-treatment or SOC treatment.

In another embodiment, the TTP is increased in a patient following treatment with a combination therapy according to the methods provided herein. In another embodiment, the PFS is increased in a patient following treatment with a combination therapy according to the methods provided herein. In one embodiment provided herein a patient is diagnosed having a CR following treatment with a combination therapy according to the methods provided herein. In one embodiment provided herein a patient is diagnosed having a PR following treatment with a combination therapy according to the methods provided herein. In one embodiment provided herein a patient is diagnosed having SD following treatment with a combination therapy according to the methods provided herein.

In some of these embodiments, the patient has locally advanced or metastatic breast cancer (e.g., histologically or cytologically confirmed) not amenable to curative therapy (e.g., surgical or radiation therapy with curative intent).

In some embodiments, the patient has progression of disease during adjuvant endocrine treatment or within 12 months of completing adjuvant endocrine therapy (e.g., with an aromatase inhibitor or tamoxifen). Non-limiting examples of matase inhibitors include anastrozole, letrozole and exemestane. In some embodiments, wherein a CDK4/6 inhibitor was included as part of neoadjuvant or adjuvant therapy, the patient has progression event must be >12 months since completion of CDK4/6 inhibitor portion of neoadjuvant or adjuvant therapy.

In one embodiment of the methods described herein, a patient has been treated with one or more cancer therapies before administration of a combination therapy described herein. In one embodiment of the methods described herein, the prior therapy comprises fulvestrant or letrozole and/or a CDK4/6 inhibitor (e.g. palbociclib, ribociclib, or abemaciclib). In another embodiment, a patient described herein has not been prior treated with fulvestrant or letrozole, a PI3K inhibitor, and/or a CDK4/6 inhibitor.

In one embodiment of the methods described herein, a patient has breast cancer described herein that is resistant to one or more cancer therapies (e.g., a CDK4/6 inhibitor such as palbociclib, ribociclib, or abemaciclib). In one embodiment of the methods described herein, resistance to cancer therapy includes recurrence of cancer or refractory cancer. Recurrence may refer to the reappearance of cancer, in the original site or a new site, after treatment. In one embodiment of the methods described herein, resistance to a cancer therapy includes progression of the cancer during treatment with the anti-cancer therapy. In some embodiments of the methods described herein, resistance to a cancer therapy includes cancer that does not response to treatment. The cancer may be resistant at the beginning of treatment or it may become resistant during treatment. In some embodiments of the methods described herein, the cancer is at early stage or at late stage.

Co-administration of GDC-0077 with a CDK4/6 inhibitor (e.g. palbociclib, ribociclib, or abemaciclib) and an endocrine therapy (e.g., fulvestrant or letrozole) may prevent or delay development of resistance of a tumor (e.g., breast cancer) to a CDK4/6 inhibitor (e.g. palbociclib, ribociclib, or abemaciclib) or a combination of a CDK4/6 inhibitor (e.g. palbociclib, ribociclib, or abemaciclib) and an endocrine therapy (e.g., fulvestrant or letrozole). Thus provided is a method of preventing or delaying development of resistance of a tumor (e.g., breast cancer) to a therapy containing a CDK4/6 inhibitor (e.g. palbociclib, ribociclib, or abemaciclib), comprising administering a combination therapy detailed herein. In some embodiments, provided is a method of preventing or delaying development of resistance of a tumor (e.g., breast cancer) to a therapy containing palbociclib, comprising administering a combination therapy comprising GDC-0077, palbociclib and fulvestrant, or a combination therapy comprises GDC-0077, palbociclib and letrozole. In some embodiments, the combination therapy is administered according to any methods as detailed herein.

In one embodiment, a patient described herein has been pretreated with an aromatase inhibitor (e.g., anastrozole, letrozole or exemestane) or tamoxifen prior to administration of the combination therapy described herein. In one such embodiment, the patient relapsed during prior treatment with an aromatase inhibitor or tamoxifen or otherwise demonstrated disease progression after such administration. In one such embodiment, the relapse or disease progression was observed during the first 12 months of an adjuvant endocrine therapy. In one embodiment, the prior treatment was with one or more aromatase inhibitors as described herein. In another embodiment, the prior treatment was with tamoxifen. In still another such embodiment, the prior treatment was for locally advanced or metastatic breast cancer. In one such embodiment, a patient described herein has been pretreated with letrozole, tamoxifen, anastrozole, or exemestane. In another such embodiment, a patient described herein has been treated for 3-6 years with an aromatase inhibitor or tamoxifen prior to administration of a combination therapy described herein. In another such embodiment, a patient described herein has been treated for greater than 6 years with an aromatase inhibitor or tamoxifen prior to administration of a combination therapy described herein. In still another embodiment, a patient herein is post-menopausal. In another embodiment, a patient herein has at least one measurable lesion as measured by, for example, RECIST.

In one embodiment of the methods described herein, a patient having hormone receptor positive and HER2 negative locally advanced or metastatic breast cancer as described herein may have undergone surgical treatment such as, for example, surgery that is breast-conserving (i.e., a lumpectomy, which focuses on removing the primary tumor with a margin), or more extensive (i.e., mastectomy, which aims for complete removal of all of the breast tissue) prior to administration of a combination therapy described herein. In another embodiment, a patient described herein may undergo surgical treatment following treatment with a combination therapy described herein.

Radiation therapy is typically administered post-surgery to the breast/chest wall and/or regional lymph nodes, with the goal of killing microscopic cancer cells left post-surgery. In the case of a breast conserving surgery, radiation is administered to the remaining breast tissue and sometimes to the regional lymph nodes (including axillary lymph nodes). In the case of a mastectomy, radiation may still be administered if factors that predict higher risk of local recurrence are present. In some embodiments of the methods provided herein a patient having hormone receptor positive and HER2 negative locally advanced or metastatic breast cancer as described herein may have received radiation therapy prior to administration of a combination therapy described herein. In other embodiments of the methods provided herein a patient having hormone receptor positive and HER2 negative locally advanced or metastatic breast cancer as described herein may have received radiation therapy following administration of a combination therapy described herein.

In another embodiment, the patient has not been pretreated with a PI3K inhibitor. In still another embodiment, the patient has not been pretreated with a mTOR inhibitor. In still another embodiment, the patient has not been pretreated with an AKT inhibitor. In yet another embodiment, the patient has not been previously treated with a cytotoxic chemotherapy regimen for metastatic breast cancer. In still another embodiment, a patient described herein has not been previously treated with a SERD (selective estrogen receptor degrader), including for example, fulvestrant.

Also provided herein are methods of inhibiting tumor growth or producing tumor regression in a patient described herein by administering a combination therapy described herein.

In one embodiment provided herein is a method of producing or improving tumor regression in a patient described herein by administering a combination therapy described herein.

The development of combination treatments poses challenges including, for example, the selection of agents for combination therapy that may lead to improved efficacy while maintaining acceptable toxicity. One particular challenge is the need to distinguish the incremental toxicity of the combination. In one embodiment of the methods described herein the combination therapy described herein (e.g. GDC-0077, fulvestrant, and palbociclib) is administered in a dosing regimen comprising a staggered dosing schedule. In one embodiment, the combination therapy described herein (e.g. GDC-0077, fulvestrant, and palbociclib) is administered simultaneously on a 28-day cycle.

In one embodiment of the methods provided herein, GDC-0077 is administered QD on each day of each 28-day cycle and palbociclib is administered QD on days 1-21 of each 28-day cycle. In such embodiments, fulvestrant is administered as described herein such as on day 1 and 15 of the first 28-day cycle and day 1 of each 28-day cycle thereafter.

In one embodiment of the methods provided herein, GDC-0077 and letrozole are each administered QD in each 28-day cycle and palbociclib is administered QD on days 1-21 of each 28-day cycle.

In some embodiments, the patient has adequate hematologic and organ function within 14 days prior to initiation of study treatment.

In patients at-risk or prone to developing hyperglycemia (for example, obese or pre-diabetic patients), metformin may be administered to manage hyperglycemia in the patient. Thus in some embodiments, provided is a method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising GDC-0077, a CDK4/6 inhibitor (e.g. palbociclib, ribociclib, or abemaciclib) and an endocrine therapy (e.g., fulvestrant or letrozole), wherein the patient has been previously treated with metformin. In some embodiments, the method comprises administering metformin, GDC-0077, palbociclib and fulvestrant, wherein GDC-0077, palbociclib and fulvestrant are administered according to any methods detailed herein. In some embodiments, the method comprises administering metformin, GDC-0077, palbociclib and letrozole, wherein GDC-0077, palbociclib and letrozole are administered according to any methods detailed herein. In some of these embodiments, the dose or regimen of metformin is adjusted to moderate, stabilize, or diminish hyperglycemia in the patient prior to administration of GDC-0077. In some of these embodiments, the patient is administered from 500 mg to 2000 mg (e.g., 500 mg) metformin daily for about 15 days before administration of GDC-0077. In some of these embodiments, the patient is administered from 500 mg to 2000 mg (e.g., 500 mg) metformin daily for about 15 days before administration of palbociclib and fulvestrant or letrozole, followed by administration of GDC-0077. In some of these embodiments, GDC-0077, palbociclib and fulvestrant or letrozole are administered according to a dosing regimen as detailed herein.

### Biomarkers

Breast cancer is a heterogeneous disease with many distinct subtypes as defined by molecular signatures and a diverse array of mutational profiles. In one embodiment, a patient can be tested for PIK3CA/AKT1/PTEN-alteration status. In one embodiment, a patient described herein can be tested for one or more of a phosphatase and tensin homolog (PTEN) mutation, loss of PTEN expression, a phosphatidylinositol-4,5-bisphosphate 3-kinase catalytic subunit alpha (PIK3CA) mutation, a protein kinase B alpha (AKT1) mutation, or a combination thereof. In one embodiment, the loss of PTEN expression is hemizygous or homozygous. In another embodiment, samples of patients described herein can be assessed for additional biomarkers in an effort to identify factors that may correlate with the safety and efficacy of the study treatments.

In one embodiment of the methods described herein, NGS, whole genome sequencing (WGS), other methods, or a combination thereof can be used for DNA obtained from blood samples and tumor tissue from patients described herein. Such samples may be analyzed to identify germline (e.g., BRCA1/2) and somatic alterations that are predictive of response to study drug, are associated with progression to a more severe disease state, are associated with acquired resistance to study drug, or can increase the knowledge and understanding of disease biology. In another embodiment of the methods described herein, patients described herein can have cancer characterized by activation of PI3K/Akt signaling such as activating mutations in PIK3CA or AKT1 as well as through alterations in PTEN, such as those provided herein. In another embodiment, PIK3CA/AKT1/PTEN-altered tumor status will be determined using an NGS assay (e.g., Foundation Medicine, Inc. [FMI]). Review of PIK3CA/AKT1/PTEN-altered status in archival tissue and response measures can be performed on an ongoing basis. Expression of biomarkers (e.g. PTEN) as provided herein can be measured using techniques known in the art such as, for example, immunohistochemistry (IHC).

Circulating tumor DNA (ctDNA) can be detected in the blood of cancer patients with epithelial cancers and may have diagnostic and therapeutic significance (Schwarzenbach et al. 2011). For example, the mutational status of tumor cells may be obtained through the isolation of ctDNA (Maheswaran S, et al. N Engl J Med 2008; 359:366-77), and ctDNA has been used to monitor treatment effectiveness in melanoma (Shinozaki M, et al. Clin Cancer Res 2007; 13:2068-74). Blood samples from patients described herein can be collected at screening, at time of first tumor assessment, and/or at the study completion/early termination visit. In one embodiment, the samples are used to evaluate oncogenic genetic alterations at baseline and to assess for the possible emergence of new alteration after treatment with GDC-0077, a CDK4/6 inhibitor (e.g. palbociclib, ribociclib, or abemaciclib), and fulvestrant or letrozole.

### EXAMPLES

### Abbreviations

AEs, adverse events;
AUC₀₋₂₄, area under the concentration-time curve at 0-24 hours;
BMI: body mass index;
CDK4/6i, cyclin-dependent kinase 4/6 inhibitor;
CI, confidence interval;
Cₘₐₓ, maximum serum concentration;
CR, complete response;
ctDNA: circulating tumor DNA;
D, day;
ECOG, Eastern Cooperative Oncology Group;
GMR, geometric mean ratio;
HbA1c, glycated haemoglobin;
HER2, human epidermal growth factor receptor 2;
MAF, mutant allele frequency;
MBC, metastatic breast cancer;
MTD, maximum tolerated dose;
NCI-CTCAE, National Cancer Institute Common Terminology Criteria for Adverse Events;
PD, pharmacodynamics;
PrD, progressive disease;
PIK3CA, phosphatidylinositol-4,5-bisphosphate 3-kinase, catalytic subunit alpha;
PK, pharmacokinetics;
PR, partial response;
pts: patients
RECIST, Response Evaluation Criteria in Solid Tumors;
SD, stable disease;
SLD, sum of longest diameters;
TRAEs, treatment-related adverse events.

### Example 1 Efficacy of GDC-0077, Palbociclib and Fulvestrant in Xenograft Mouse Model

Efficacy of GDC-0077, palbociclib and fulvestrant alone and in combination was conducted in the MCF-7 (PIK3CA mutant E545K, ER+) breast carcinoma xenograft model using female athymic nude mice.

### Procedures:

Implant 0.36 mg estradiol pellets, s.c. between the scapulae, 1-3 days prior to cell implantation.

Set up CR female NCr nu/nu mice with 1×10⁷ MCF7 tumor cells in 0% Matrigel sc in mammary fat pad.

Cell Injection Volume is 0.1 mL/mouse.

Mice are monitored for signs of estrogen toxicity. Monitoring includes bladder palpation once a week for the first 3-4 weeks after pellet implantation. After 3-4 weeks the animals are monitored a minimum of twice a week, since signs of toxicity may develop. Monitoring consists of bladder palpation to determine if the bladders are enlarged. When enlarged, bladders are expressed. When animals cannot urinate on their own, their bladders cannot be expressed, or when there is blood or sediment in the urine, they are euthanized.

Age at Start Date: 8 to 10 weeks.

Perform a pair match when tumors reach an average size of 180-220 mm³, and begin treatment.

Body Weight: 5/2 then biwk to end

Caliper Measurement: biwk to end

Report any adverse reactions or death to RM, SD, RD or Site Director immediately.

Any individual animal with a single observation of >than 30% body weight loss or three consecutive measurements of >25% body weight loss are euthanized.

Stop dosing in any group with a mean body weight loss of >20% or >10% mortality. The group is not euthanized and recovery is allowed. Within a group with >20% weight loss, individuals hitting the individual body weight loss endpoint will be euthanized. When the group treatment related body weight loss is recovered to within 10% of the original weights, dosing may resume at a lower dose or less frequent dosing schedule. Exceptions to non-treatment body weight % recovery may be allowed on a case-by-case basis.

Endpoint TGD. Animals are monitored individually. The endpoint of the experiment is a tumor volume of 2000 mm³ or 45 days, whichever comes first. Responders can be followed longer. When the endpoint is reached, the animals are euthanized per SOP.

### Dosing Solution Preparation

Solution of GDC-0077 in 0.5% Methylcellulose: 0.2% Tween 80 in DI Water was prepared every day at room temperature and stored at 4° C.

Solution of palbociclib in 0.5% Methylcellulose: 0.2% Tween 80 in DI Water was prepared every week at room temperature and stored at 4° C.

Solution of fulvestrant in 10% Ethanol in Corn Oil was prepared every week at room temperature and stored at 4° C.

The vehicle used was 0.5% Methylcellulose: 0.2% Tween 80 in DI Water.

### Dosing:

Allow the formulation to equilibrate to room temperature prior to dosing.

Dose for GDC-0077 were determined by MTD.

Dose palbociclib, then fulvestrant, then GDC-0077 (immediately right after each other). On days when fulvestrant is not dosed, palbociclib is dosed first, immediately followed by GDC-0077.

Dosing volume for GDC-0077, palbociclib, and the vehicle was 10 mL/kg (0.200 mL/20 g mouse), adjusted accordingly for body weight.

Dosing volume for fulvestrant was 0.2 mL/mouse, not adjusted for body weight.

### Results

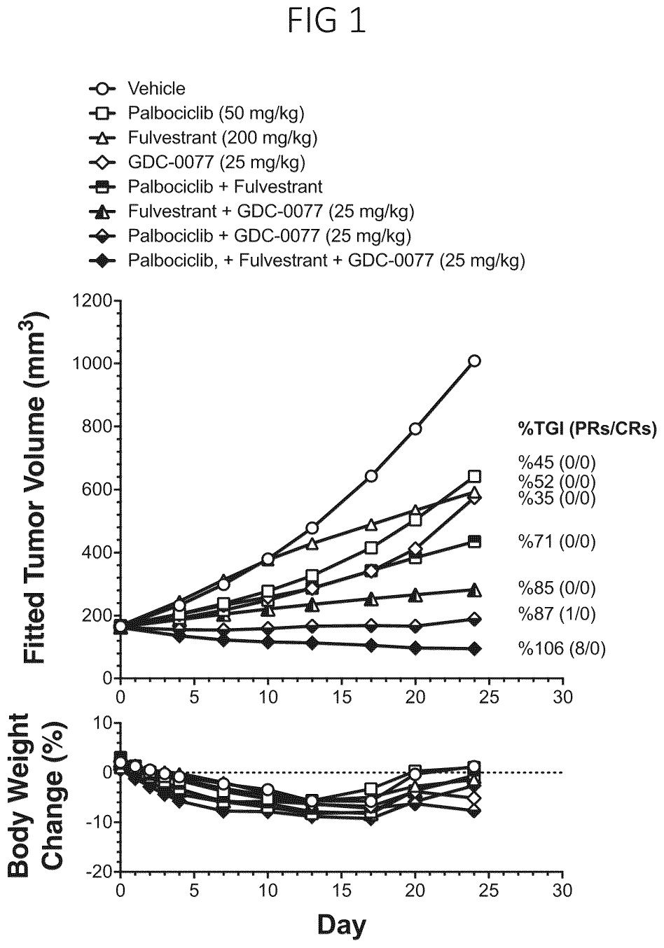

GDC-0077 and palbociclib were dosed once daily by oral gavage, and fulvestrant was dosed once weekly subcutaneously (s.c.) at 200 mg/kg (5 mg/mouse). In one set of experiments, each mouse (N=12) received 25 mg/kg (oral, QD) GDC-0077 (G) for 21 days, 50 mg/kg (oral, QD) palbociclib (P) for 21 days, and 200 mg/kg (s.c., weekly) fulvestrant (F) for 3 weeks. The effects of each single agent (P, F, and G), double combinations (P+F, G+F, and G+P) and the triple combination (G+P+F) are shown in FIG. 1. Tumor growth inhibition (% TGI), CR and PRs are listed in Table 1. The results show that GDC-0077 enhances the efficacy of palbociclib and fulvestrant in MCF-7 PIK3CA-mutant E545K ER+breast carcinoma xenograft mouse model. The triple combination also showed more PRs. All drug treatments and combinations were tolerated based on body weights.

TABLE 1
% TGI, CR and PRs in MCF-7 mouse xenograft
(groups of 12 animals)

| Agents | % TGI | PR | CR |
|---|---|---|---|
| P | 45 | 0 | 0 |
| F | 52 | 0 | 0 |
| G | 35 | 0 | 0 |
| P + F | 71 | 0 | 0 |
| G + F | 85 | 0 | 0 |
| G + P | 87 | 1 | 0 |
| G + P + F | 106 | 8 | 0 |

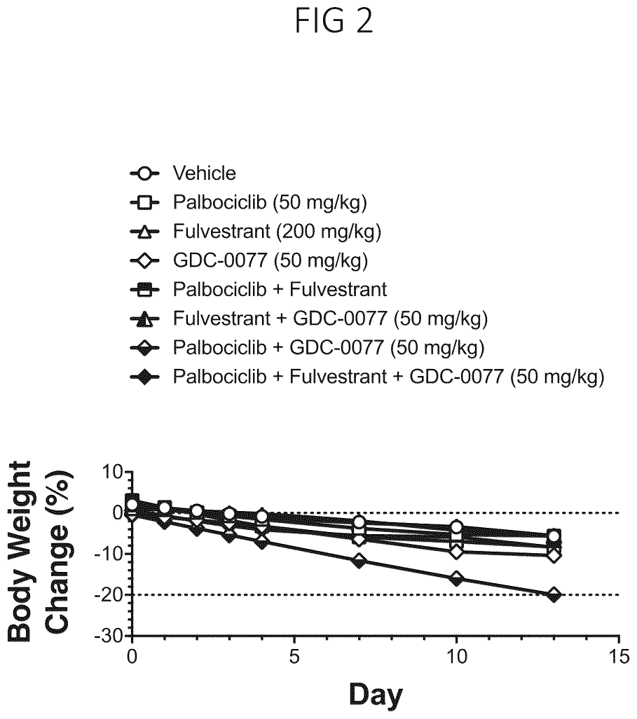

However, at 50 mg/kg daily dose of GDC-0077, the triple combination resulted in toxicity as shown by the significant body weight loss (FIG. 2).

### Example 2 Dose Escalation Study of GDC-0077

A Phase I dose-escalation study of oral daily GDC-0077 monotherapy and combined with other therapies was conducted in patients with locally advanced or metastatic PIK3CA-mutant solid tumors.

### Study Design

Open-label, Phase I dose escalation with 3+3 design.

GDC-0077 was administered daily (QD) orally at 6, 9, or 12 mg.

The primary objective was to determine the maximum tolerated dose (MTD) and/or Recommended Phase II Dose (RP2D), and assess the safety of GDC-0077 in patients with PIK3CA-mutant solid tumors.

Key eligibility criteria: PIK3CA-mutation in tumor tissue or ctDNA; fasting glucose ≤140 mg/dL; HbA1c<7%.

To evaluate single dose pharmacokinetics (PK), frequent blood sampling was performed up to 48 hours after the initial dose at Cycle 1 Day 1. Once daily dosing was initiated from Cycle 1 Day 8 and blood sampling to evaluate PK at steady state was conducted at Cycle 1 Day 15.

Tumor assessments were performed by RECIST v1.1 at screening and every 8 weeks to assess preliminary anti-tumor activity. Clinical benefit rate was defined as complete or partial response, or stable disease lasting ≥24 weeks.

Pharmacodynamic activity was assessed pre- and on-study (after 2 weeks of daily GDC-0077 treatment) with FDG-PET scans, in tumor biopsies via immunohistochemistry (IHC), and in ctDNA samples (by FoundationACT).

### Results

As of the clinical cut-off, 20 patients were enrolled and treated with single-agent GDC-0077. All patients were female, with hormone-receptor positive, HER2-negative (HR+/HER2−) breast cancer, except for 1 male patient with colorectal cancer. Median age was 65 years (41-77) and 11 patients (55%) had ECOG 0 at baseline. Five patients (25%) were obese (BMI ≥30) overall, while 2 patients (50%) were obese in the 12-mg cohort.

The median number of prior cancer therapies in the metastatic setting was 3 (1-12). Fifteen (75%) patients were previously treated with chemotherapy in the metastatic setting.

The median GDC-0077 treatment duration was 3.8 months (range 1.1-17.5) and GDC-0077 cumulative dose intensity was 97%.

All patients discontinued from treatment due to disease progression (radiographic or clinical).

### Results—Safety

The most frequent adverse events (AEs) and treatment-related (TR) AEs are shown in Table 2.

TABLE 2
Adverse events in ≥20% patients overall

| | GDC-0077 6 mg | | GDC-0077 9 mg | | GDC-0077 12 mg | | All Patients | |
|---|---|---|---|---|---|---|---|---|
| | (n = 7) | | (n = 9) | | (n = 4) | | (N = 20) | |
| | All | Related | All | Related | All | Related | All | Related |
| Diarrhea | 7 | 3 | 4 | 3 | 3 | 2 | 14 | 8 |
| | (100%) | (43%) | (44%) | (33%) | (75%) | (50%) | (70%) | (40%) |
| Hyperglycemia | 4 | 4 | 6 | 6 | 4 | 4 | 14 | 14 |
| | (57%) | (57%) | (67%) | (67%) | (100%) | (100%) | (70%) | (70%) |
| Nausea | 2 | 1 | 5 | 1 | 4 | 1 | 11 | 3 |
| | (29%) | (14%) | (56%) | (11%) | (100%) | (25%) | (55%) | (15%) |
| Vomiting | 2 | 1 | 5 | 3 | 2 | — | 9 | 4 |
| | (29%) | (14%) | (56%) | (33%) | (50%) | | (45%) | (20%) |
| Fatigue | — | — | 4 | 2 | 2 | 1 | 6 | 3 |
| | | | (44%) | (22%) | (50%) | (25%) | (30%) | (15%) |
| AST increased | 1 | — | 4 | — | — | — | 5 | — |
| | (14%) | | (44%) | | | | (25%) | |
| Constipation | 1 | 1 | 2 | 1 | 2 | — | 5 | 2 |
| | (14%) | (14%) | (22%) | (11%) | (50%) | | (25%) | (10%) |
| Decreased appetite | 2 | 1 | 3 | 3 | — | — | 5 | 4 |
| | (29%) | (14%) | (33%) | (33%) | | | (25%) | (20%) |
| Hyponatremia | 1 | — | 2 | 1 | 2 | 1 | 5 | 2 |
| | (14%) | | (22%) | (11%) | (50%) | (25%) | (25%) | (10%) |
| Thrombocytopenia | 2 | 1 | 3 | 1 | — | — | 5 | 2 |
| | (29%) | (14%) | (33%) | (11%) | | | (25%) | (10%) |
| Abdominal pain | — | — | 2 | 1 | 2 | — | 4 | 1 |
| | | | (22%) | (11%) | (50%) | | (20%) | (5%) |
| Alopecia | — | — | 4 | 3 | — | — | 4 | 3 |
| | | | (44%) | (33%) | | | (20%) | (15%) |
| ALT increased | 1 | — | 3 | — | — | — | 4 | — |
| | (14%) | | (33%) | | | | (20%) | |
| Alkaline phos. incr. | — | — | 2 | — | 2 | — | 4 | — |
| | | | (22%) | | (50%) | | (20%) | |
| Dyspnea | 1 | — | 3 | — | — | — | 4 | — |
| | (14%) | | (33%) | | | | (20%) | |
| Hypophosphatemia | — | — | 3 | — | 1 | — | 4 | — |
| | | | (33%) | | (25%) | | (20%) | |
| Peripheral edema | 1 | — | 3 | 1 | — | — | 4 | 1 |
| | (14%) | | (33%) | (11%) | | | (20%) | (5%) |
| Stomatitis¹ | 1 | 1 | 3 | 3 | — | — | 4 | 4 |
| | (14%) | (14%) | (33%) | (33%) | | | (20%) | (20%) |
| Vision blurred | 2 | 1 | 1 | — | 1 | 1 | 4 | 2 |
| | (29%) | (14%) | (11%) | | (25%) | (25%) | (20%) | (10%) |

¹Stomatitis grouped term = stomatitis, mucosal inflammation, mouth ulceration, glossitis, lip ulceration, palatal ulcer, and tongue ulceration.

Dose-limiting toxicities occurred in 2 patients at 12 mg (1 Grade 4 hyperglycemia, 1 Grade 3 fatigue that lasted 5 days). The MTD of GDC-0077 was established at 9 mg QD.

Grade ≥3 TRAEs were hyperglycemia (4 patients, 20%), and lymphopenia, fatigue, nausea, weight loss, and asthenia (1 patient, 5% each).

AEs resulted in dose reduction in 6 patients (30%; included 3 patients treated at the 12 mg dose level, which exceeded MTD), and included hyperglycemia and nausea.

Hyperglycemia was the most frequent TRAE and was manageable with oral anti-hyperglycemic medications (most commonly metformin in 11 patients). Stomatitis (including stomatitis, mucosal inflammation, mouth ulceration, glossitis, lip ulceration, palatal ulcer, and tongue ulceration; all Grade 1) generally responded to topical corticosteroid treatment (i.e. dexamethasone mouth wash). Rash (including rash, maculo-papular rash, dermatitis acneiform, erythema, and generalized rash) occurred in 3 patients (15%) (1 unrelated Grade 2, otherwise all Grade 1). No treatment related high-grade gastrointestinal toxicities were reported (treatment-related diarrhea events were all Grade 1-2). No colitis was reported (9 patients treated ≥5 months on study treatment).

### Results—Pharmacokinetics

Plasma exposure of GDC-0077 increased proportionally following single and multiple doses.

Mean half-life (t_{1/2}) following a single dose of GDC-0077 was 18.1 hours.

With continuous once-daily dosing, there was ˜1.3- to 3.1 fold accumulation consistent with the observed t_{1/2} and the dosing frequency.

PK variability was low for Cₘₐₓ and AUC₀₋₂₄ after a single dose as well as at steady state at MTD (9 mg) (% CV ˜20%).

### Results—Clinical Activity

Overall, partial response (PR) was observed in 5 patients (best overall response rate: 25%, all responders had at least 3 lines of prior metastatic therapy), with confirmed PR in 4 patients (confirmed overall response rate: 20%).

Among the 19 patients with measurable disease, the best overall response rate was 26%, and the confirmed overall response rate was 21%.

The clinical benefit rate was 45% (9 of 20 patients).

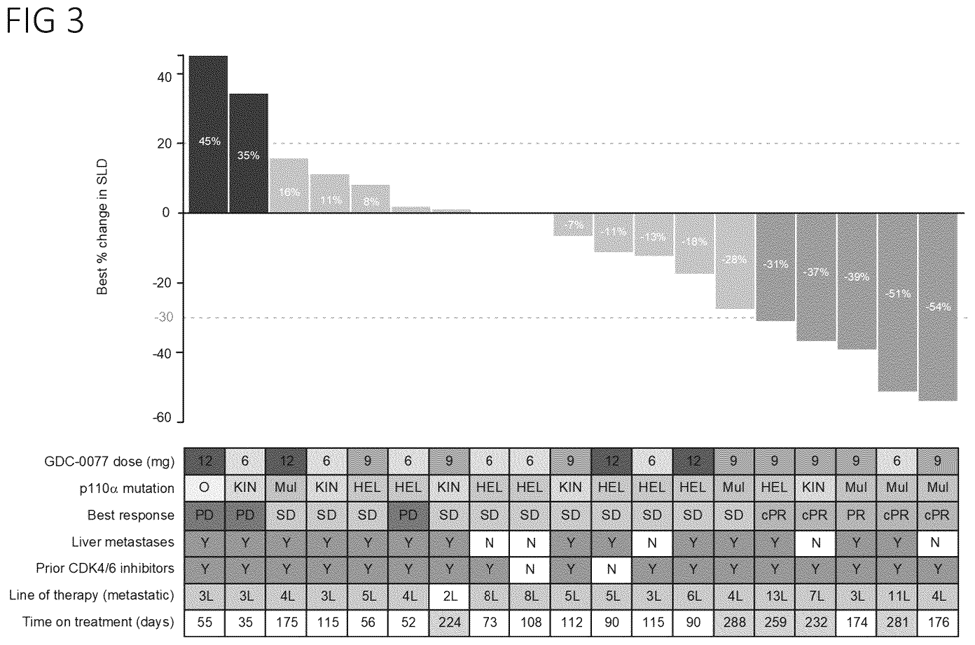

The single agent antitumor activity is shown in FIG. 3. p110α mutation: KIN=kinase domain (H1047, M1043); HEL=helical domain (E545, E542, Q546); Mul=multiple mutations; O=other: N345K.

### Results—Pharmacodynamics

FDG-PET scans at baseline and after 2 weeks of daily GDC-0077 showed metabolic responses at all dose levels evaluated.

Decreased PI3K pathway activity was observed in paired tumor biopsies assessed by IHC for phosphoproteins.

Decreased ctDNA PIK3CA mutant allele frequency was observed over time in the majority of specimens.

### Conclusions

The single agent dose escalation study of the oral p110α-selective and mutant-degrading inhibitor GDC-0077 demonstrated a manageable safety profile and identified the MTD at 9 mg once daily.

PK analysis showed a linear PK profile and supported daily dosing.

Anti-tumor activity showed promising preliminary results with an overall response rate of 210% in patients with tumors harboring PIK3CA mutations.

Pharmacodynamic modulation was observed in tumors by FDG-PET, in tumor biopsies by IHC, and in ctDNA dynamics.

### Example 3 Clinical Studies with GDC-0077/Palbociclib Letrozole Combination

An open-label, Phase I dose escalation study of oral daily GDC-0077 alone and in combination with endocrine and targeted therapies is underway.

### Study Design

GDC-0077 administered daily (QD) orally at 6 or 9 mg with letrozole (G+L) or 3, 6, or 9 mg in combination with palbociclib and letrozole (G+P+L).

Letrozole administered QD orally at 2.5 mg. Palbociclib administered QD orally at 125 mg on Days 1-21, followed by 7 days off, in 28-day cycles. Both administered as per dosing labels.

Key eligibility criteria: PIK3CA-mutation in tumor tissue or ctDNA; fasting glucose ≤140 mg/dL, HbA1c<7%. For dose expansion, prior CDK4/6 inhibitor was prohibited (G+P+L arm only) and up to 1 prior metastatic chemotherapy allowed (both arms).

Tumor assessments at screening and every 8 weeks by RECIST v1.1 to evaluate anti-tumor activity per investigator assessment. Clinical benefit rate (CBR) defined as complete or partial response, or stable disease lasting ≥24 weeks.

Pharmacodynamic activity was assessed pre- and on-study (after 2 weeks of daily GDC-0077 treatment), in tumor biopsies via immunohistochemistry (IHC), and in ctDNA samples (by FoundationACT).

### Results

As of the clinical cut-off, 37 patients enrolled in G+L and 33 patients in G+P+L.

All patients were post-menopausal females with HR+/HER2− breast cancer. Median age was 58 years (43-79) in G+L, and 57 years (37-80) in G+P+L. ECOG of 0 was reported in 25 patients (68%) in G+L, and in 20 patients (61%) in G+P+L. Eight patients were enrolled in G+L (22%) and 10 patients in G+P+L (30%) with BMI ≥30 (obese).

Median number of prior cancer therapies in the metastatic setting was 3 (0-11) in G+L, and 2 (0-4) in G+P+L.

Number of patients treated with prior chemotherapy in the metastatic setting: 18 patients (49%) in G+L, and 14 patients (42%) in G+P+L.

Median GDC-0077 treatment duration was 3.7 months (range 0.2-17.3) in the G+L arm and 11.5 months (range 1.3-23.9) in the G+P+L arm, with GDC-0077 cumulative dose intensity of 98% for both arms. Letrozole cumulative dose intensity was 100% in G+L and 99% in G+P+L. Palbociclib cumulative dose intensity was 86% in G+P+L.

Fifty-one of 70 patients had discontinued treatment primarily due to disease progression; 1 due to Grade 3 hyperglycemia in the G+P+L arm (no discontinuation due to AE in G+L).

### Results—Safety

The most frequent treatment-related (TR) AEs and Grade 3-4 TRAEs are shown in Table 3 for G+L and in Table 4 for G+P+L.

TABLE 3
TRAEs in >10% of patients and corresponding Grade 3-4 TRAEs for
GDC-0077 + letrozole (N = 37)

| | All Grades | Grades 3-4 |
|---|---|---|
| Total no. pts. with ≥1 AE | 35 (95%) | 11 (30%) |
| Hyperglycemia | 25 (68%) | 7 (19%) |
| Nausea | 14 (38%) | — |
| Stomatitis¹ | 11 (30%) | — |
| Diarrhea | 10 (27%) | — |
| Dysgeusia | 9 (24%) | — |
| Fatigue | 8 (22%) | 2 (5%) |
| Decreased appetite | 7 (19%) | — |
| Dry mouth | 6 (16%) | — |
| Vomiting | 5 (14%) | — |
| Arthralgia | 4 (11%) | — |

¹Stomatitis grouped term = stomatitis, mucosal inflammation, mouth ulceration, glossitis, lip ulceration, palatal ulcer, and tongue ulceration.

TABLE 4
TRAEs in >10% of patients and corresponding Grade 3-4
TRAEs for GDC-0077 + palbociclib + letrozole (N = 33)

| | All Grades | Grades 3-4 |
|---|---|---|
| Total no. pts. with ≥1 AE | 33 (100%) | 27 (82%) |
| Neutropenia | 26 (79%) | 21 (64%) |
| Stomatitis¹ | 22 (67%) | 1 (3%) |
| Hyperglycemia | 18 (55%) | 6 (18%) |
| Anemia | 17 (52%) | 1 (3%) |
| Diarrhea | 15 (46%) | — |
| Alopecia | 14 (43%) | — |
| Nausea | 13 (39%) | — |
| Thrombocytopenia | 13 (39%) | 2 (6%) |
| Decreased appetite | 12 (36%) | — |
| Dysgeusia | 9 (27%) | — |
| Rash² | 9 (27%) | — |
| Vomiting | 8 (24%) | — |
| Muscle spasms | 7 (21%) | — |
| Dry mouth | 7 (21%) | 1 (3%) |
| Asthenia | 6 (18%) | — |
| Leukopenia | 6 (18%) | 3 (9%) |
| Fatigue | 4 (12%) | — |
| Abdominal pain | 4 (12%) | — |
| Weight decreased | 4 (12%) | — |
| Peripheral edema | 4 (12%) | — |
| Dry skin | 4 (12%) | — |
| GERD | 4 (12%) | — |
| Lymphopenia | 4 (12%) | 3 (9%) |

¹Stomatitis grouped term = stomatitis, mucosal inflammation, mouth ulceration, glossitis, lip ulceration, palatal ulcer, and tongue ulceration.
²Rash grouped term = rash, maculo-papular rash, dermatitis acneiform, erythema, and generalized rash.

No Grade 4 TRAEs were reported in G+L arm; Grade 4 TRAEs included neutropenia (6 patients, 18%) and hypophosphatemia (1 patient, 3%) in G+P+L arm.

No dose-limiting toxicities reported in either combination with GDC-0077 doses up to 9 mg QD.

Hyperglycemia was manageable with oral anti-hyperglycemic medication. It resulted in GDC-0077 dose interruptions in 9 (24%) patients and GDC-0077 dose reductions in 2 (5%) patients in the G+L arm, and in 8 (24%) patients and in 1 (3%) patient in the G+P+L arm.

Neutropenia was similar to published data from studies with palbociclib+endocrine therapy (Cristofanilli et al, Lancet Oncol 2016). It resulted in palbociclib dose interruptions in 10 (30%) patients and palbociclib dose reductions in 12 (36%) patients.

Stomatitis (including stomatitis, mucosal inflammation, mouth ulceration, glossitis, lip ulceration, palatal ulcer, and tongue ulceration) responded to treatment with dexamethasone mouthwash.

Rash (including rash, maculo-papular rash, dermatitis acneiform, erythema, and generalized rash) occurred in 4 patients (11%) in the G+L arm (related in 1 patient [3%]); and in 11 patients (33%) in the G+P+L arm (related in 9 patients [27%]). All were Grade 1.

### Results—Clinical Activity

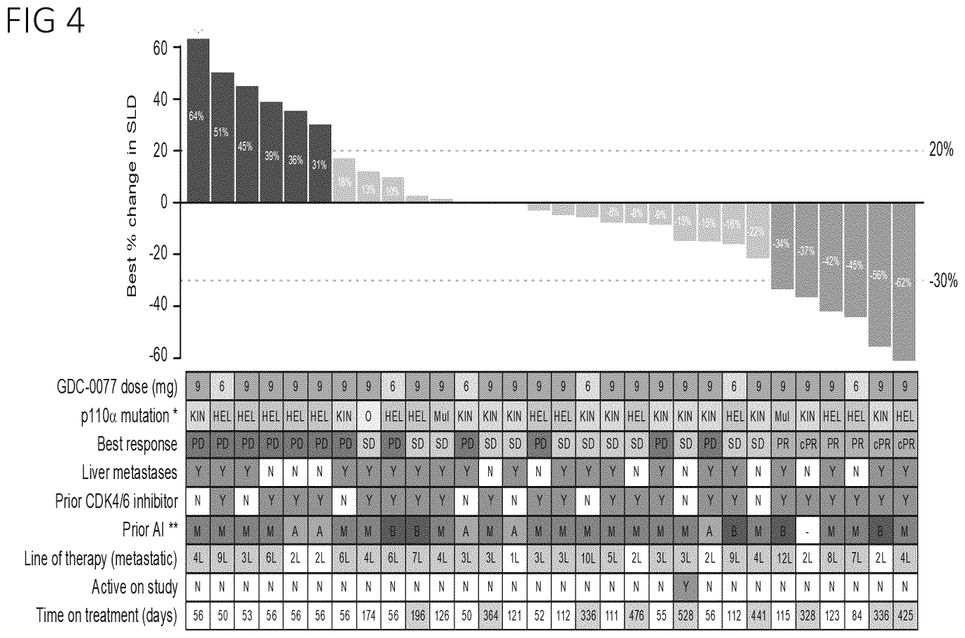

In the G+L arm, PR was reported in 6 patients (best overall response rate: 16%), confirmed PR in 3 patients (confirmed ORR: 8%) and CBR in 13 patients (35%) (FIG. 4). * p110α mutation: KIN=kinase domain (H1047, M1043); HEL=helical domain (E545, E542, Q546); Mul=multiple mutations; O=other: N345K. ** Prior AI=prior aromatase inhibitor; A=adjuvant; M=metastatic setting, B=both adjuvant and metastatic. Purple squares indicate >6 months on treatment.

Among the 31 patients with measurable disease, the best overall response rate was 19% and the confirmed ORR was 10% (1 patient did not have a post-baseline tumor assessment).

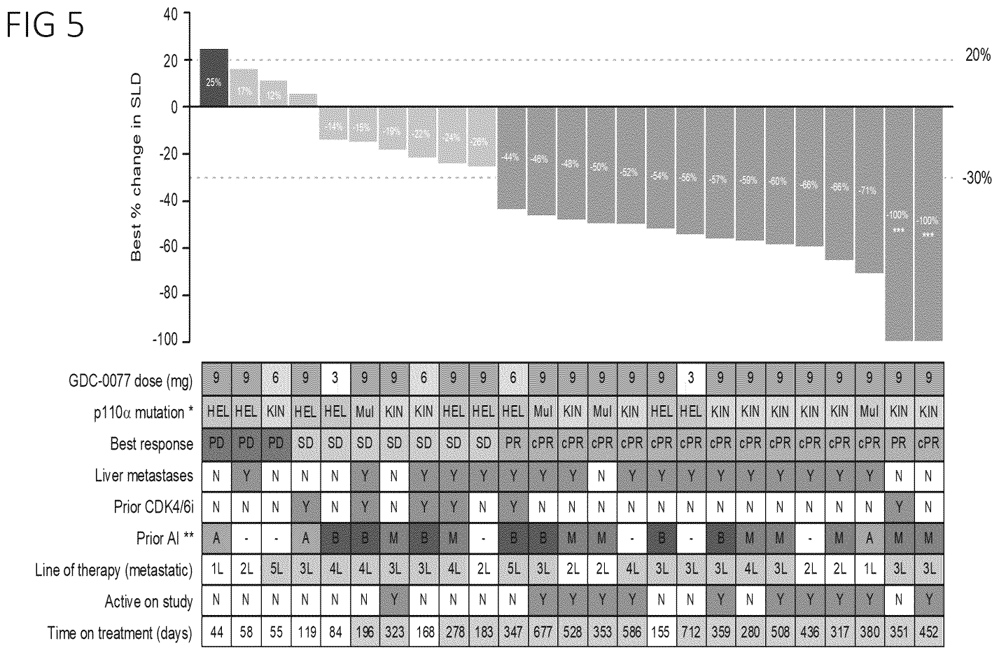

In the G+P+L arm, PR was reported in 15 patients (best overall response rate: 46%), confirmed PR in 13 patients (confirmed ORR: 39%), and CBR in 26 patients (79%) (FIG. 5). * p110α mutation: KIN=kinase domain (H1047, M1043); HEL=helical domain (E545, E542, Q546); Mul=multiple mutations; O=other: N345K. ** Prior AI=prior aromatase inhibitor; A=adjuvant; M=metastatic setting, B=both adjuvant and metastatic. Purple squares indicate >6 months on treatment.

Among the 25 patients with measurable disease, the best overall response rate was 60% and the confirmed ORR was 52%.

### Results—Pharmacokinetics

Preliminary results indicated that plasma exposure of GDC-0077 is similar between single agent and combination arms in this study.

Similarly, the pharmacokinetics of palbociclib and letrozole administered in combination with GDC-0077 are comparable to single-agent PK of these agents reported in literature.

There was no drug-drug interaction between GDC-0077 and the concomitant medications (palbociclib and letrozole) administered in this study.

### Results—Pharmacodynamics

Robust PD downregulation of PI3K pathway effectors (pAKT, pS6) was observed in paired tumor biopsies.

PIK3CA mutant allele frequency by ctDNA was decreased between Cycle 1 Day 1 and Cycle 1 Day 15 in the majority of available samples.

### Conclusions

This Phase Ib study of GDC-0077 in combination with letrozole with and without palbocicilib demonstrated a manageable safety profile combining GDC-0077 at its single agent recommended Phase II dose of 9 mg with letrozole and palbociclib at standard doses.

No PK DDI was observed for GDC-0077, palbociclib or letrozole when administered in combination compared to their single agent PK.

Promising preliminary anti-tumor activity was observed at GDC-0077 doses of 3, 6, and 9 mg, and in both combinations, with an ORR of 10% in G+L arm and 52% in G+P+L arm.

Pharmacodynamic modulation was observed in tumor biopsies by IHC and in ctDNA dynamics.

### Example 4 Clinical Studies with GDC-0077 Fulvestrant Combination

An open-label, Phase I/Ib study of oral daily GDC-0077 in combination with fulvestrant is underway.

### Study Design

GDC-0077 was administered orally at 9 mg once daily on Days 1-28 of each 28-day cycle. Fulvestrant (F) was adminstered intramuscularly at 500 mg on Days 1 and 15 of Cycle 1, and on Day 1 of each subsequent cycles until intolerable toxicity or disease progression.

Safety (NCI-CTCAE v4), PK, and preliminary antitumor activity (clinical benefit rate [CBR]: RECIST v1.1 stable disease for ≥24 weeks, partial response [PR], or complete response) were assessed. The effect of a standard high-fat meal on the PK of G was evaluated after a single dose and at steady state. Relevant signaling and pharmacodynamic (PD) biomarkers were assessed using circulating tumor (ct) DNA samples.

### Results

As of the clinical cut-off, 20 patients were enrolled in the food-effect portion of the G+F arm.

All patients were post-menopausal females with HR+/HER2− breast cancer. Median age 54.5 years (range: 31-85); 17 patients (85%) with ECOG 0; seven patients (35%) with BMI ≥30 kg/m2 and/or HbA1c≥5.7% (Eligibility criteria required HbA1c<7%); 15 patients (75%) with ≥two prior therapy lines for metastatic breast cancer; nine patients (45%) previously treated with one prior chemotherapy in the metastatic setting.

### Safety

Among the 20 pts at this cutoff, the median GDC-0077 treatment duration was 5.9 months (range 1.7-17.8); cumulative dose intensity, 98%. Adverse events (AEs) led to dose reduction in 3 pts (15%).

The most common treatment-related (TR) AEs (≥4 pts, 20%) were hyperglycemia (11, 55%), diarrhea (10, 50%), stomatitis (Grouped terms: stomatitis, mucosal inflammation, and mouth ulceration; 9, 45%), nausea (8, 40%), decreased appetite (7, 35%), dysgeusia, fatigue, and muscle spasms (4 each, 20%).

Grade ≥3 TRAEs were hyperglycemia, nausea, lymphopenia, hyperamylasemia, and hyperlipasemia (1 each, 5%).

### Pharmacokinetics

The PK of GDC-0077 in combination with fulvestrant was similar to single-agent PK. Comparable GDC-0077 exposures (Cₘₐₓ and AUC₀₋₂₄) were observed following administration in fasted or fed states.

### Clinical Activity

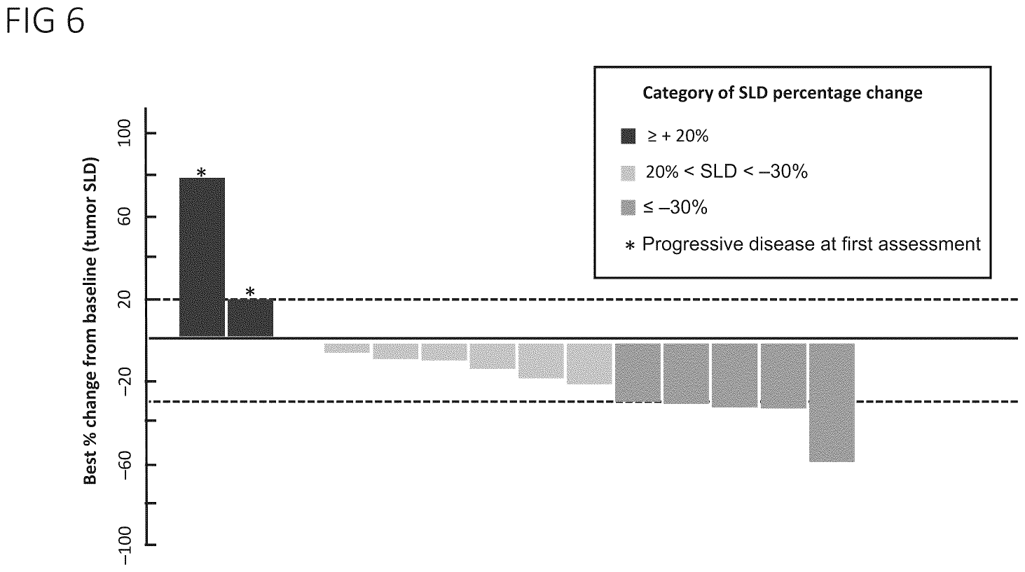

Seventeen pts (85%) discontinued treatment, all due to radiographic/clinical disease progression. Overall, 5/14 pts with measurable disease had a PR (36%; 2 received prior F; 4, prior CDK4/6i), of whom 2 pts (14%) had a confirmed PR (FIG. 6). The clinical benefit rate (CBR) was 60% ( 12/20 pts).

### Pharmacodynamics

Most patients showed decreased ctDNA PIK3CA-mutant allele frequency during treatment (paired ctDNA data not shown).

### Conclusions

GDC-0077 plus fulvestrant demonstrated a manageable safety profile, similar PK to GDC-0077 alone, preliminary antitumor activity, and PD modulation of PIK3CA mutation allele frequency in ctDNA. The presence of food did not impact the rate or extent of GDC-0077 absorption significantly following single or at steady state.

### Example 5 Phase 1 Clinical Studies with Inavolisib/Palbociclib/Fulvestrant Combination

A Phase I/Ib study of inavolisib (GDC-0077) alone and combined with endocrine therapy+palbociclib is ongoing (NCT03006172) in patients with HR+/HER2−, PIK3CA mutant breast cancer.

### Study Design

Inavolisib 9 mg oral once daily+palbociclib 125 mg 21/28 days+fulvestrant 500 mg intramuscularly on Day 1 (and Day 15 of Cycle 1) of 28-day cycles, were administered in Arms E and F, until intolerable toxicity or disease progression.

In Arm F, patients were obese and/or pre-diabetic (body mass index ≥30 kg/m² and/or hemoglobin A1c≥5.7%). The patients also received metformin ≤2000 mg daily, starting at 500 mg at Cycle 1, Day 1, prior to initiating inavolisib at Cycle 1, Day 15 instead of Day 1 as in Arm E.

Additional key eligibility criteria included pre-/post-menopausal status, PIK3CAmut tumors as per local or central tumor testing, Eastern Cooperative Oncology Group (ECOG) performance status 0-1, no prior PI3K or CDK4/6 inhibitor (CDK4/6i) therapy, and ≤1 prior chemotherapy for Arm E (no restrictions on prior CDK4/6i therapy or chemotherapy for Arm F). Pts with diabetes requiring medication or hemoglobin A1c>7% were excluded.

PIK3CAmut allele frequency was assessed in circulating tumor (ct)DNA from serial plasma collections using FoundationACT™ (Cambridge, MA).

### Patients

As of the clinical cut-off, 36 pts were enrolled: 20 in Arm E and 16 in Arm F. Enrollment was ongoing in Arm F. Baseline characteristics are shown in Table 5.

TABLE 5
Patient characteristics and treatment exposure

| | Arm E | | Arm F | |
|---|---|---|---|---|
| | (n = 20) | | (n = 16) | |
| Median age, years (range) | 55 | (33-73) | 65 | (33-73) |
| Median body mass index, kg/m² (range) | 25 | (19.2-38.0) | 33 | (28.4-42.1) |
| ECOG performance status 0, n (%) | 10 | (50%) | 8 | (50%) |
| ≥2 prior lines of therapy for mBC, n (%) | 5 | (25%) | 13 | (81%) |
| Prior fulvestrant, n (%) | 3 | (15%) | 12 | (75%) |
| Prior CDK4/6 inhibitor, n (%) | 0 | | 10 | (63%) |
| Median inavolisib treatment duration, | 6.8 | (1.1-17.7) | 6.3 | (1.2-15.3) |
| months (range) | | | | |

| | | |
|---|---|---|
| Median cumulative inavolisib | 93% | 91% |
| dose intensity | | |
| Median cumulative palbociclib | 86% | 95% |
| dose intensity | | |
| Median cumulative fulvestrant | 100% | 100% |
| dose intensity | | |

Sixteen pts (44%) discontinued treatment: 14 due to radiographic disease progression (five in Arm E, nine in Arm F); one due to an adverse event (AE; treatment-related Grade 2 panniculitis in Arm F); and one withdrew (Arm F).

### Safety

Grade 1 rash (grouped term, defined as: rash, rash maculo-papular, dermatitis acneiform) was reported in two pts (10%) in Arm E and one pt (6.3%) in Arm F.

Hyperglycemia was managed with antihyperglycemic agents in eight pts (40%) in Arm E and nine (56%) in Arm F (in addition to metformin), and with inavolisib dose modifications in five pts (25%) in Arm E and nine (56%) in Arm F. Three pts (19%) in Arm F required an inavolisib dose reduction (none in Arm E). Grade 3-4 hyperglycemia was observed in seven pts (44%) in Arm F despite pre-treatment with metformin in obese and/or pre-diabetic pts (Table 6).

Stomatitis (grouped term) was reported in 67% of pts ( 24/36) across both arms and managed in the majority with dexamethasone mouthwash (used as treatment and not as prophylaxis in the vast majority of cases).

Neutropenia was managed with palbociclib dose modifications (interruptions and/or reductions) in 12 pts (60%) in Arm E and two (13%) in Arm F. Three pts (15%) in Arm E required palbociclib dose reductions (none in Arm F).

No grade 5 AEs were reported in either arm.

TABLE 6
Treatment-related AEs (except those related to metformin) in
≥4 pts in either arm, and corresponding treatment-related grade 3-4 AEs

| | Arm E | | Arm F | |
|---|---|---|---|---|
| | (n = 20) | | (n = 16) | |
| | All Grades | Grades 3-4 | All Grades | Grades 3-4 |
| Total # of patients | 20 (100%) | 16 (80%) | 14 (88%) | 12 (75%) |
| with ≥1 AE (%) | | | | |
| Neutropenia | 17 (85%) | 13 (65%) | 9 (56%) | 9 (56%) |
| Stomatitis* | 16 (80%) | 2 (10%) | 8 (50%) | 0 |
| Hyperglycemia | 12 (60%) | 3 (15%) | 11 (69%) | 7 (44%) |
| Diarrhea | 9 (45%) | 1 (5%) | 8 (50%) | 0 |
| Thrombocytopenia^{†} | 9 (45%) | 4 (20%) | 3 (19%) | 1 (6%) |
| Anemia | 7 (35%) | 1 (5%) | 4 (25%) | 2 (13%) |
| Nausea | 5 (25%) | 0 | 8 (50%) | 0 |
| Decreased appetite | 5 (25%) | 0 | 4 (25%) | 0 |
| Fatigue | 5 (25%) | 0 | 3 (19%) | 1 (6%) |
| Alopecia | 4 (20%) | 0 | 3 (19%) | 0 |
| Asthenia | 4 (20%) | 0 | 0 | 0 |
| Vision blurred | 0 | 0 | 4 (25%) | 0 |
| Dyspepsia | 0 | 0 | 4 (25%) | 0 |

*Stomatitis (grouped term) was defined as: glossodynia, mucositis, mucosal inflammation, mouth ulceration, lip ulceration.
^{†}Thrombocytopenia (grouped term) was defined as: thrombocytopenia, decreased platelet count.

### Clinical Activity

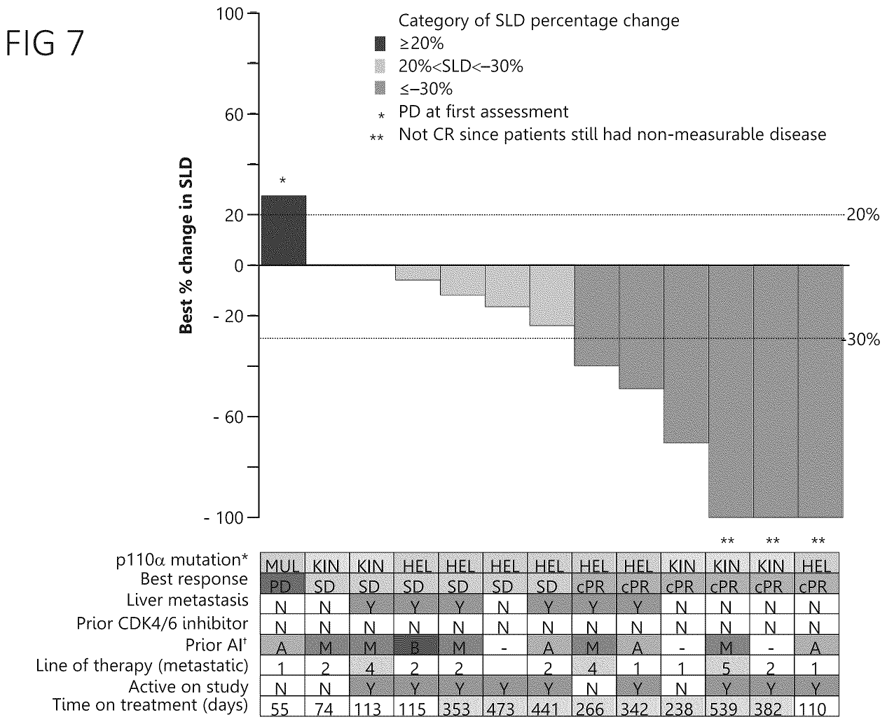

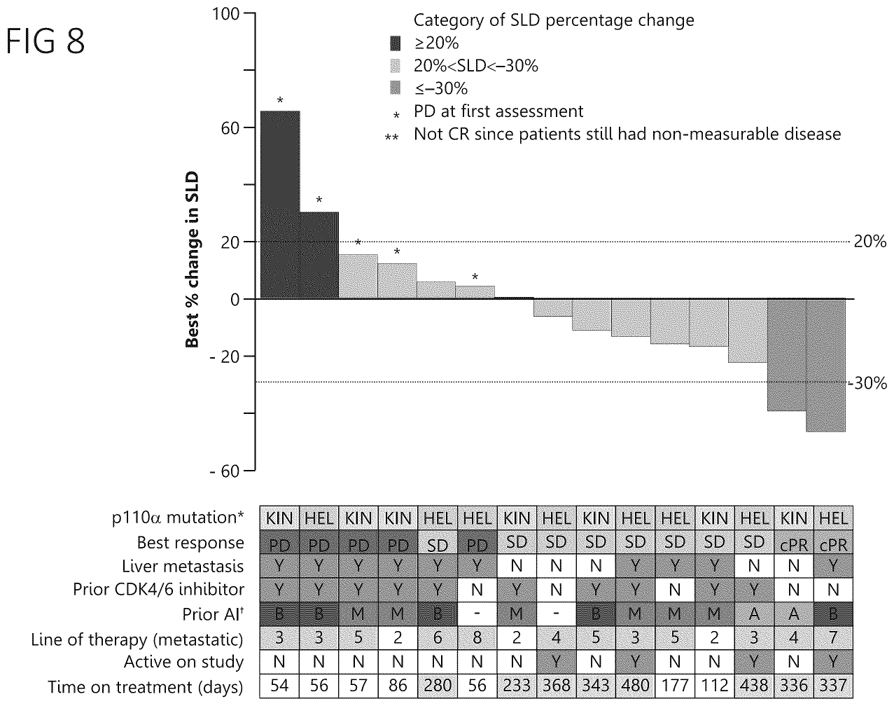

Waterfall graph of antitumor activity in Arm E and Arm F are shown in FIG. 7 and FIG. 8 respectively. Overall, among patients with measurable disease: Six of 15 pts (40%) had a partial response (PR) in Arm E; 2/15 pts (13%) had a PR in Arm F (both received prior fulvestrant). All were confirmed PR. Clinical benefit rate (defined as stable disease for ≥24 weeks, PR, or complete response [CR]) was 58% ( 21/36 pts: 12 in Arm E; nine in Arm F). One patient in Arm E with evaluable-only disease at baseline had a CR.

### Pharmacokinetics

The pharmacokinetics of inavolisib in combination with palbociclib+fulvestrant was similar to that of single-agent inavolisib.

There were no drug-drug interactions between inavolisib and concomitant therapies (palbociclib and fulvestrant [and metformin]) administered.

### Pharmacodynamics

There were limited data from ctDNA analysis of PIK3CAmut allele frequency; however, decreases of PIK3CAmut allele frequency were observed over time in some patients experiencing stable disease or a PR.

### Conclusions

This phase I/Ib study demonstrated a manageable safety profile when combining inavolisib at its single-agent recommended dose of 9 mg with palbociclib+fulvestrant at standard doses, with no unexpected safety signals and similar pharmacokinetics to inavolisib alone. In obese and/or pre-diabetic patients enrolled to Arm F, hyperglycemia was frequent despite initiating metformin prior to inavolisib.

Encouraging preliminary antitumor activity with a response rate of 40% was observed in Arm E (13% in Arm F). There were limited data for modulation of PIK3CAmut allele frequency.

### Example 6 Phase 3 Clinical Studies with Inavolisib Palbociclib Fulvestrant Combination

A multicenter, international, Phase III randomized, double-blind, placebo-controlled study is designed to evaluate the efficacy, safety, and pharmacokinetics of inavolisib (GDC-0077) in combination with palbociclib and fulvestrant compared with placebo plus palbociclib and fulvestrant in patients with PIK3CA-mutant, hormone receptor (HR)-positive, HER2-negative locally advanced or metastatic breast cancer whose disease progressed during treatment or within 12 months of completing adjuvant endocrine therapy and who have not received prior systemic therapy for metastatic disease.

### Arms and Interventions

Experimental Arm: GDC-0077+Palbociclib+Fulvestrant

Participants receive: (a) oral GDC-0077 on Days 1-28 of each 28-day cycle; (b) oral palbociclib on Days 1-21 of each 28-day cycle; and (c) intramuscular (IM) fulvestrant approximately every 4 weeks.

Placebo Comparator Arm: Placebo+Palbociclib+Fulvestrant

Participants receive: (a) oral placebo on Days 1-28 of each 28-day cycle; (b) oral palbociclib on Days 1-21 of each 28-day cycle; and (c) intramuscular (IM) fulvestrant approximately every 4 weeks.

### Outcome Measures

### Primary Outcome Measure:

### 1. Progression-Free Survival (PFS)

[Time Frame: From randomization to the first occurrence of disease progression or death from any cause, whichever occurs first (up to 6 years)]

### Secondary Outcome Measure:

### 2. Objective Response Rate (ORR)

[Time Frame: Up to 6 years]

### 3. Best Overall Response Rate (BOR)

[Time Frame: Up to 6 years]

### 4. Duration of Response (DOR)

[Time Frame: From the first occurrence of a CR or PR to the first occurrence of disease progression or death from any cause, whichever occurs first (up to 6 years)]

### 5. Clinical Benefit Rate (CBR)

[Time Frame: Up to 6 years]

### 6. Overall Survival (OS)

[Time Frame: From randomization to death from any cause (up to 6 years)]

### 7. Time to Deterioration (TTD) in Pain

[Time Frame: Treatment: Day 1 of Cycles 1-3, then Day 1 of every other cycle until treatment discontinuation. Posttreatment: Every 8 weeks for 2 years, then every 12 weeks thereafter, to end of study (up to 6 years)]

### 8. TTD in Physical Function

[Time Frame: Treatment: Day 1 of Cycles 1-3, then Day 1 of every other cycle until treatment discontinuation. Posttreatment: Every 8 weeks for 2 years, then every 12 weeks thereafter, to end of study (up to 6 years)]

### 9. TTD in Role Function

[Time Frame: Treatment: Day 1 of Cycles 1-3, then Day 1 of every other cycle until treatment discontinuation. Posttreatment: Every 8 weeks for 2 years, then every 12 weeks thereafter, to end of study (up to 6 years)]

### 10. TTD in Global Health Status

[Time Frame: Treatment: Day 1 of Cycles 1-3, then Day 1 of every other cycle until treatment discontinuation. Posttreatment: Every 8 weeks for 2 years, then every 12 weeks thereafter, to end of study (up to 6 years)]

11. Percentage of Participants with Adverse Events

[Time Frame: From randomization through the end of study (up to 6 years)]

### 12. Plasma Concentration of GDC-0077

[Time Frame: At pre-defined intervals from baseline to the end of study (up to 6 years)]

### 13. Plasma Concentration of Palbociclib

[Time Frame: At pre-defined intervals from baseline to the end of study (up to 6 years)]

### 14. Plasma Concentration of Fulvestrant

[Time Frame: At pre-defined intervals from baseline to the end of study (up to 6 years)]

### Eligibility

In one embodiment of the study, patients of age 18 years or older; all sex (not gender based) are accepted. Healthy volunteers are not accepted. The target population inclusion and exclusion criteria are as follows.

### Inclusion Criteria

Confirmed diagnosis of HR+/HER2− breast cancer
Metastatic or locally advanced disease not amenable to curative therapy
Progression of disease during adjuvant endocrine treatment or within 12 months of completing adjuvant endocrine therapy with an aromatase inhibitor or tamoxifen
Receiving LHRH agonist therapy for at least 2 weeks prior to Day 1 of Cycle 1 if pre/peri-menopausal
Confirmation of biomarker eligibility (detection of specified mutation(s) of PIK3CA via specified test)
Consent to provide fresh or archival tumor tissue specimen
Measurable disease per Response Evaluation Criteria in Solid Tumors, Version 1.1; “bone-only” disease, even if considered measurable, is not eligible
Eastern Cooperative Oncology Group Performance Status of 0 or 1
Life expectancy of >6 months
Adequate hematologic and organ function within 14 days prior to initiation of study treatment

### Exclusion Criteria

Metaplastic breast cancer
Any history of leptomeningeal disease or carcinomatous meningitis
Any prior systemic therapy for metastatic breast cancer
Prior treatment with fulvestrant or any selective estrogen-receptor degrader
Prior treatment with any PI3K, AKT, or mTOR inhibitor, or any agent whose mechanism of action is to inhibit the PI3K-AKT-mTOR pathway
Type 2 diabetes requiring ongoing systemic treatment at the time of study entry; or any history of Type 1 diabetes
Known and untreated, or active CNS metastases. Patients with a history of treated CNS metastases are eligible
Active inflammatory or infectious conditions in either eye, or any eye conditions expected to require surgery during the study treatment period
Symptomatic active lung disease, or requiring daily supplemental oxygen
History of inflammatory bowel disease or active bowel inflammation
Anti-cancer therapy within 2 weeks before study entry
Investigational drug(s) within 4 weeks before randomization
Prior radiotherapy to >=25% of bone marrow, or hematopoietic stem cell or bone marrow transplantation
Chronic corticosteroid therapy or immunosuppressants
Pregnant, lactating, or breastfeeding, or intending to become pregnant during the study or within 60 days after the final dose of study treatment
Major surgical procedure, or significant traumatic injury, within 28 days prior to Day 1 of Cycle 1

### Additional Embodiments A

Embodiment A1. A combination for use in treating a PIK3CA mutant, hormone receptor positive and HER2 negative locally advanced or metastatic breast cancer, wherein said combination comprises:
(i) inavolisib;
(ii) palbociclib; and
(iii)fulvestrant,
and wherein said combination is administered over a 28-day cycle.

Embodiment A2. A combination for use in treating a PIK3CA mutant, hormone receptor positive and HER2 negative locally advanced or metastatic breast cancer, wherein said combination is administered in a combination therapy comprising a dosing regimen comprising:
a. administering inavolisib QD on days 1-28 of a first 28-day cycle;
b. administering palbociclib QD on days 1-21 of a first 28-day cycle; and
c. administering fulvestrant on days 1 and 15 of a first 28-day cycle.

Embodiment A3. The combination for use according to Embodiment A2, wherein the dosing regimen further comprising one or more additional 28-day cycles comprising:
a. administering inavolisib on days 1-28 of each additional 28-day cycle;
b. administering palbociclib on days 1-21 of each additional 28-day cycle; and
c. administering fulvestrant on day 1 of each additional 28-day cycle.

Embodiment A4. The combination for use according to any one of Embodiments A1-A3, wherein inavolisib is administered at an amount of 9 mg.

Embodiment A5. The combination for use according to Embodiment A4, wherein inavolisib is administered at an amount of 9 mg in an oral tablet.

Embodiment A6. The combination for use according to any one of Embodiments A1-A5, wherein palbociclib is administered at an amount of 125 mg in an oral capsule or tablet.

Embodiment A7. The combination for use according to any one of Embodiments A1-A6, wherein fulvestrant is administered at an amount of 500 mg by intramuscular (IM) infusion.

Embodiment A8. A combination for use in treating a PIK3CA mutant, hormone receptor positive and HER2 negative locally advanced or metastatic breast cancer, wherein said combination comprises:
(i) inavolisib;
(ii) palbociclib; and
(iii)letrozole,
and wherein said combination therapy is administered over a 28-day cycle.

Embodiment A9. A combination for use in treating a PIK3CA mutant, hormone receptor positive and HER2 negative locally advanced or metastatic breast cancer, wherein said combination is administered in a combination therapy comprising a dosing regimen comprising:
a. administering inavolisib QD on days 1-28 of a first 28-day cycle;
b. administering palbociclib QD on days 1-21 of a first 28-day cycle; and
c. administering letrozole QD on days 1-28 of a first 28-day cycle.

Embodiment A10. The combination for use according to Embodiment A9, wherein the dosing regimen further comprising one or more additional 28-day cycles comprising:
a. administering inavolisib on days 1-28 of each additional 28-day cycle;
b. administering palbociclib on days 1-21 of each additional 28-day cycle; and
c. administering letrozole on days 1-28 of each additional 28-day cycle.

Embodiment A11. The combination for use according to any one of Embodiments A8-A10, wherein inavolisib is administered at an amount of 3, 6 or 9 mg.

Embodiment A12. The combination for use according to Embodiment A11, wherein inavolisib is administered at an amount of 9 mg.

Embodiment A13. The combination for use according to Embodiment A12, wherein inavolisib is administered at an amount of 9 mg in an oral tablet.

Embodiment A14. The combination for use according to any one of Embodiments A8-A13, wherein palbociclib is administered at an amount of 125 mg in an oral capsule or tablet.

Embodiment A15. The combination for use according to any one of Embodiments A8-A14, wherein letrozole is administered at an amount of 2.5 mg in an oral tablet.

Embodiment A16. A combination for use according to any one of Embodiments A1-A15 for inhibiting tumor growth or producing/increasing tumor regression in a patient having PIK3CA mutant, hormone receptor positive and HER2 negative locally advanced or metastatic breast cancer.

Embodiment A17. The combination for use according to any one of Embodiments A1-A16, wherein the patient has locally advanced or metastatic breast cancer not amenable to curative therapy.

Embodiment A18. The combination for use according to any one of Embodiments A1-A17, wherein the patient has progression of disease during adjuvant endocrine treatment or within 12 months of completing adjuvant endocrine therapy with an aromatase inhibitor or tamoxifen.

Embodiment A19. The combination for use according to any one of Embodiments A1-A18, wherein the patient has adequate hematologic and organ function within 14 days prior to initiation of study treatment.

Embodiment A20. The combination for use according to any one of Embodiments A1-A19, wherein the patient is post-menopausal.

Embodiment A21. A combination for use according to any one of Embodiments A1-A20 for preventing or delaying development of resistance of a breast cancer to a therapy containing palbociclib.

### Additional Embodiments B

Embodiment B1. Use of a combination in the manufacture of a medicament for treating a PIK3CA mutant, hormone receptor positive and HER2 negative locally advanced or metastatic breast cancer, wherein said combination comprises:
(i) inavolisib;
(ii) palbociclib; and
(iii)fulvestrant,
and wherein said combination therapy is administered over a 28-day cycle.

Embodiment B2. Use of a combination in the manufacture of a medicament for treating a PIK3CA mutant, hormone receptor positive and HER2 negative locally advanced or metastatic breast cancer, wherein said combination is administered in a combination therapy comprising a dosing regimen comprising:
a. administering inavolisib QD on days 1-28 of a first 28-day cycle;
b. administering palbociclib QD on days 1-21 of a first 28-day cycle; and
c. administering fulvestrant on days 1 and 15 of a first 28-day cycle.

Embodiment B3. The use of Embodiment B2, wherein the dosing regimen further comprising one or more additional 28-day cycles comprising:
a. administering inavolisib on days 1-28 of each additional 28-day cycle; b. administering palbociclib on days 1-21 of each additional 28-day cycle; and
c. administering fulvestrant on day 1 of each additional 28-day cycle.

Embodiment B4. The use of any one of Embodiments B1-B3, wherein inavolisib is administered at an amount of 9 mg.

Embodiment B5. The use of Embodiment B4, wherein inavolisib is administered at an amount of 9 mg in an oral tablet.

Embodiment B6. The use of any one of Embodiments B1-B5, wherein palbociclib is administered at an amount of 125 mg in an oral capsule or tablet.

Embodiment B7. The use of any one of Embodiments B1-B6, wherein fulvestrant is administered at an amount of 500 mg by intramuscular (IM) infusion.

Embodiment B8. Use of a combination in the manufacture of a medicament for treating a PIK3CA mutant, hormone receptor positive and HER2 negative locally advanced or metastatic breast cancer, wherein said combination comprises:
(i) inavolisib;
(ii) palbociclib; and
(iii)letrozole,
and wherein said combination is administered over a 28-day cycle.

Embodiment B9. Use of a combination for the manufacture of a medicament for treating a PIK3CA mutant, hormone receptor positive and HER2 negative locally advanced or metastatic breast cancer, wherein said combination is administered in a combination therapy comprising a dosing regimen comprising:
a. administering inavolisib QD on days 1-28 of a first 28-day cycle;
b. administering palbociclib QD on days 1-21 of a first 28-day cycle; and
c. administering letrozole QD on days 1-28 of a first 28-day cycle.

Embodiment B10. The use of Embodiment B9, wherein the dosing regimen further comprising one or more additional 28-day cycles comprising:
a. administering inavolisib on days 1-28 of each additional 28-day cycle;
b. administering palbociclib on days 1-21 of each additional 28-day cycle; and
c. administering letrozole on days 1-28 of each additional 28-day cycle.

Embodiment B11. The use of any one of Embodiments B8-B10, wherein inavolisib is administered at an amount of 3, 6 or 9 mg.

Embodiment B12. The use of Embodiment B11, wherein inavolisib is administered at an amount of 9 mg.

Embodiment B13. The use of Embodiment B12, wherein inavolisib is administered at an amount of 9 mg in an oral tablet.

Embodiment B14. The use of any one of Embodiments B8-B13, wherein palbociclib is administered at an amount of 125 mg in an oral capsule or tablet.

Embodiment B15. The use of any one of Embodiments B8-B14, wherein letrozole is administered at an amount of 2.5 mg in an oral tablet.

Embodiment B16. Use of a combination in the manufacture of a medicament for inhibiting tumor growth or producing/increasing tumor regression in a patient having PIK3CA mutant, hormone receptor positive and HER2 negative locally advanced or metastatic breast cancer, according to the combination or use of any one of Embodiments B1-B15.

Embodiment B17. The use of any one of Embodiments B1-B16, wherein the patient has locally advanced or metastatic breast cancer not amenable to curative therapy.

Embodiment B18. The use of any one of Embodiments B1-B17, wherein the patient has progression of disease during adjuvant endocrine treatment or within 12 months of completing adjuvant endocrine therapy with an aromatase inhibitor or tamoxifen.

Embodiment B19. The use of any one of Embodiments B1-B18, wherein the patient has adequate hematologic and organ function within 14 days prior to initiation of study treatment.

Embodiment B20. The use of any one of Embodiments B1-B19, wherein the patient is post-menopausal.

Embodiment B21. Use of a combination in the manufacture of a medicament for preventing or delaying development of resistance of a breast cancer to a therapy containing palbociclib, according to the combination or use of any one of Embodiments B1-B20.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

We claim:

1. A method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising:
(i) inavolisib;
(ii) palbociclib; and
(iii) fulvestrant,
wherein said combination therapy is administered over a 28-day cycle.

2. A method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising a dosing regimen comprising:
a. administering inavolisib QD on days 1-28 of a first 28-day cycle;
b. administering palbociclib QD on days 1-21 of a first 28-day cycle; and
c. administering fulvestrant on days 1 and 15 of a first 28-day cycle.

3. The method of claim 1 or 2, further comprising one or more additional 28-day cycles comprising:
a. administering inavolisib on days 1-28 of each additional 28-day cycle;
b. administering palbociclib on days 1-21 of each additional 28-day cycle; and
c. administering fulvestrant on day 1 of each additional 28-day cycle.

4. The method of any one of claims 1-3, wherein inavolisib is administered at an amount of 9 mg.

5. The method of claim 4, wherein inavolisib is administered at an amount of 9 mg in an oral tablet.

6. The method of any one of claims 1-5, wherein palbociclib is administered at an amount of 125 mg in an oral capsule or tablet.

7. The method of any one of claims 1-6, wherein fulvestrant is administered at an amount of 500 mg by intramuscular (IM) infusion.

8. A method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising:
(i) inavolisib;
(ii) palbociclib; and
(iii) letrozole,
wherein said combination therapy is administered over a 28-day cycle.

9. A method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising a dosing regimen comprising:
a. administering inavolisib QD on days 1-28 of a first 28-day cycle;
b. administering palbociclib QD on days 1-21 of a first 28-day cycle; and
c. administering letrozole QD on days 1-28 of a first 28-day cycle.

10. The method of claim 8 or 9, further comprising one or more additional 28-day cycles comprising:
a. administering inavolisib on days 1-28 of each additional 28-day cycle;
b. administering palbociclib on days 1-21 of each additional 28-day cycle; and
c. administering letrozole on days 1-28 of each additional 28-day cycle.

11. The method of any one of claims 8-10, wherein inavolisib is administered at an amount of 3, 6 or 9 mg.

12. The method of claim 11, wherein inavolisib is administered at an amount of 9 mg.

13. The method of claim 12, wherein inavolisib is administered at an amount of 9 mg in an oral tablet.

14. The method of any one of claims 8-13, wherein palbociclib is administered at an amount of 125 mg in an oral capsule or tablet.

15. The method of any one of claims 8-14, wherein letrozole is administered at an amount of 2.5 mg in an oral tablet.

16. A method of inhibiting tumor growth or producing/increasing tumor regression in a patient having PIK3CA mutant, hormone receptor positive and HER2 negative locally advanced or metastatic breast cancer, the method comprising administering to the patient a combination therapy according to the methods of any one of claims 1-15.

17. The method of any one of claims 1-16, wherein the patient has locally advanced or metastatic breast cancer not amenable to curative therapy.

18. The method of any one of claims 1-17, wherein the patient has progression of disease during adjuvant endocrine treatment or within 12 months of completing adjuvant endocrine therapy with an aromatase inhibitor or tamoxifen.

19. The method of any one of claims 1-18, wherein the patient has adequate hematologic and organ function within 14 days prior to initiation of study treatment.

20. The method of any one of claims 1-19, wherein the patient is post-menopausal.

21. A method of preventing or delaying development of resistance of a breast cancer to a therapy containing palbociclib, said method comprising administering a combination therapy comprising inavolisib, palbociclib and fulvestrant, or a combination therapy comprises inavolisib, palbociclib and letrozole, according to the method of any one of claims 1-20.
